(19) United States
(12) Patent Application Publication
(10) Pub. No.: US 2025/0367009 A1
(43) Pub. Date: Dec. 4, 2025

(54) STENT TRAPPED FLUID REMOVAL

(71) Applicant: EDWARDS LIFESCIENCES CORPORATION, Irvine, CA (US)

(72) Inventors: Ben Shitrit, Eilat (IL); Gideon Meyer-Brodnitz, Ramot Menashe (IL); Gil Senesh, Adi (IL); Roei Mayo, Irvine, CA (US); Yuval Kasher, Kfar Shmuel (IL)

(21) Appl. No.: 19/306,765

(22) Filed: Aug. 21, 2025

Related U.S. Application Data
(63) Continuation of application No. PCT/US2024/019149, filed on Mar. 8, 2024.
(60) Provisional application No. 63/489,850, filed on Mar. 13, 2023.

(51) Int. Cl. A61F 2/89; A61M 1/00; A61M 25/10

(52) U.S. Cl. CPC A61F 2/89; A61M 1/743; A61M 1/76; A61M 25/1002; A61M 2025/1052; A61M 2202/0413; A61M 2210/12

(57) ABSTRACT

A stent implant includes a first stent segment having a non-circular axial cross-sectional shape in a relaxed configuration thereof, second and third stent segments on opposite sides of the first stent segment, the second and third stent segments having a circular relaxed axial cross-sectional shape, a fluid-tight covering disposed on at least one of an outer diameter or an inner diameter of the first, second, and third stent segments, and a blood flow egress channel in fluid communication with a space on the outer diameter of the first stent segment, the blood flow egress channel configured to permit blood flow to pass axially out of the space through the blood flow egress channel.

### RELATED APPLICATION(S)

This application is a continuation of International Patent Application No. PCT/US24/19149, filed Mar. 8, 2024, which claims the benefit of U.S. Provisional Patent Application No. 63/489,850, filed on Mar. 13, 2023, the complete disclosures of which are hereby incorporated by reference in their entireties.

### BACKGROUND

The present disclosure generally relates to the field of medical implant devices, including stent implant devices. Stent implant devices can be designed for intravascular deployment. Blood stagnation between stent implant devices and blood vessel tissue, such as in gaps formed between stent frames and blood vessel walls, can affect patient outcomes.

### SUMMARY

Described herein are devices, methods, and systems relating to non-circular stent devices/assemblies including blood expulsion and/or aspiration components/elements. Such components/elements can facilitate configured the conforming of blood vessel walls around stent walls of a non-circular segment of a stent implant device as otherwise-trapped blood is evacuated from a space/area between the stent walls and the blood vessel walls. Blood expulsion and/or aspiration components/elements associated with the present disclosure can include blood-flow tubes, valves, and/or combinations of the same. Expansion devices, such as tubular balloons, can be used to expand non-circular stent segments to cause blood expulsion, which may be through tube and/or valve components in some examples. For stent implant devices that include fluid-tight coverings, blood expulsion/aspiration features as disclosed herein can reduce the risk of blood stagnation radially outside of the stent.

For purposes of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

Any of the example methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1 shows example cardiac and vascular anatomy.

FIGS. 2A and 2B show side and axial cross-sectional views, respectively, of a compliant blood vessel experiencing compliant expansion and contraction over a cardiac cycle.

FIG. 3 shows an example stiff aorta.

FIGS. 4-1 and 4-2 show a blood vessel in circular and non-circular axial cross-sectional shapes, respectively.

FIGS. 5A and 5B show perspective and axial views, respectively, of a non-circular stent disposed in a blood vessel.

FIGS. 6A and 6B show perspective and axial views, respectively, of a non-circular stent disposed in a blood vessel in accordance with one or more examples.

FIGS. 7A-7D show perspective, side, and axial views, respectively, of a stent having circular end portions and a non-circular medial portion in accordance with one or more examples.

FIGS. 8A-8D show perspective, side, and axial views, respectively, of a stent having circular end portions and a non-circular medial portion in accordance with one or more examples.

FIGS. 9A and 9B show perspective and axial views, respectively, of a stent having oval (non-peanut) end portions and a peanut-shaped medial portion in accordance with one or more examples.

FIGS. 10A and 10B show side and axial views, respectively, of a stent implant device implanted within a blood vessel with trapped blood collected around portion(s) of the stent implant in accordance with one or more examples.

FIGS. 11A and 11B show side and axial views, respectively, of a stent implant device including fluid aspiration/expulsion tube(s) in accordance with one or more examples.

FIGS. 12A and 12B show side and axial views, respectively, of a stent implant device having blood passed through fluid aspiration/expulsion tube(s) associated therewith in accordance with one or more examples.

FIGS. 13A and 13B show side and axial views, respectively, of a stent implant device including fluid expulsion valve(s) in accordance with one or more examples.

FIGS. 14A and 14B show side and axial views, respectively, of a stent implant device having blood passed through expulsion valve(s) associated therewith in accordance with one or more examples.

FIGS. 15A and 15B show side and axial views, respectively, of a stent implant device having associated therewith a fluid expulsion balloon in accordance with one or more examples.

FIGS. 16A and 16B show side and axial views, respectively, of a stent implant device having a fluid expulsion balloon expanded therein in accordance with one or more examples.

### DETAILED DESCRIPTION

The headings provided herein are for convenience only and do not necessarily affect the scope or meaning of the claimed invention.

Although certain preferred examples are disclosed below, it should be understood that the inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices. components, aspects, features, modules, or systems identified by the same reference number in another figure. Furthermore, aspects of separate figures identified with common reference numbers can be interpreted to share characteristics or to be entirely independent of one another.

Where an alphanumeric reference identifier is used that comprises a numeric portion and an alphabetic portion (e.g., ‘10a,’ ‘10’ is the numeric portion and ‘a’ is the alphabetic portion), references in the written description to only the numeric portion (e.g., ‘10’) may refer to any feature identified in the figures using such numeric portion (e.g., ‘10a,’ ‘10b,’ ‘10c,’ etc.), even where such features are identified with reference identifiers that concatenate the numeric portion thereof with one or more alphabetic characters (e.g., ‘a,’ ‘b,’ ‘c,’ etc.). That is, a reference in the present written description to a feature ‘10’ may be understood to refer to either an identified feature ‘10a’ in a particular figure of the present disclosure or to an identifier ‘10’ or ‘10b’ in the same figure or another figure, as an example.

Certain standard anatomical terms of location are used herein to refer to the anatomy of animals, and namely humans, with respect to various examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

### Vascular Anatomy and Compliance

Certain examples are disclosed herein in the context of vascular implant devices, and in particular, implant devices comprising non-circular segments having tissue-engagement elements associated therewith, wherein such implant devices are implanted in the aorta. However, although certain principles disclosed herein may be particularly applicable to the anatomy of the aorta, it should be understood that stent implant devices having tissue-engagement elements in accordance with the present disclosure may be implanted in, or configured for implantation in, any suitable or desirable blood vessels or other anatomy, such as the inferior vena cava.

The anatomy of the heart and vascular system is described below to assist in the understanding of certain inventive concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves.

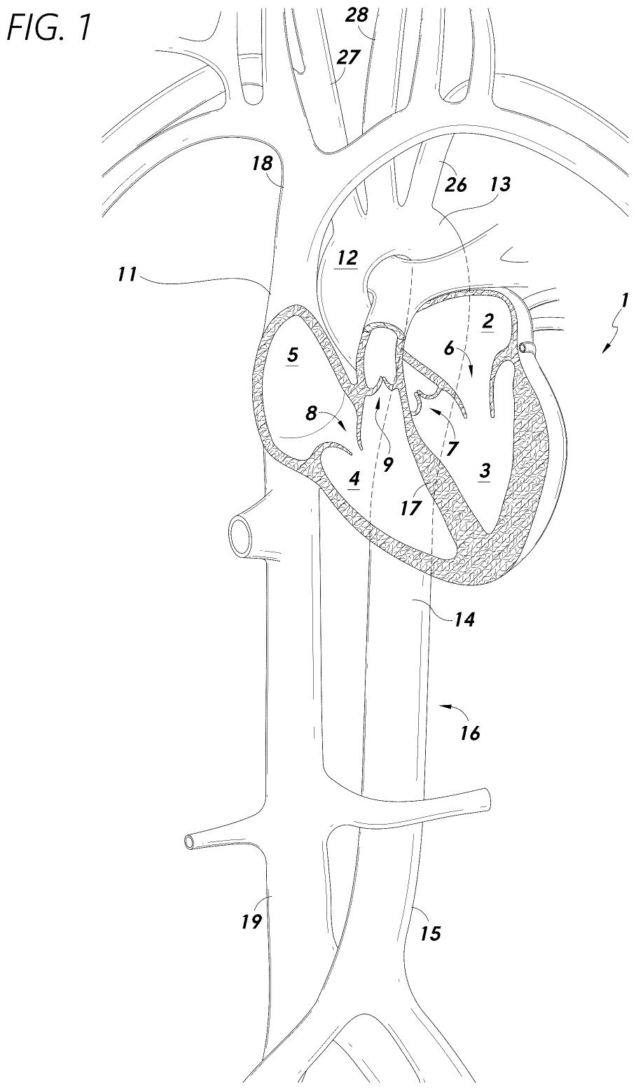

FIG. 1 illustrates an example representation of a heart 1 and associated vasculature having various features relevant to one or more examples of the present inventive disclosure. The heart 1 includes four chambers, namely the left atrium 2, the left ventricle 3, the right ventricle 4, and the right atrium 5. In terms of blood flow, blood generally flows from the right ventricle 4 into the pulmonary artery via the pulmonary valve 9, which separates the right ventricle 4 from the pulmonary artery 11 and is configured to open during systole so that blood may be pumped toward the lungs and close during diastole to prevent blood from leaking back into the heart from the pulmonary artery 11. The pulmonary artery 11 carries deoxygenated blood from the right side of the heart to the lungs. The pulmonary artery 11 includes a pulmonary trunk and left and right pulmonary arteries that branch off of the pulmonary trunk, as shown.

The tricuspid valve 8 separates the right atrium 5 from the right ventricle 4. The tricuspid valve 8 generally has three cusps/leaflets and may generally close during ventricular contraction (i.e., systole) and open during ventricular expansion (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3. A wall of muscle 17, referred to as the septum, separates the left 2 and right 5 atria and the left 3 and right 4 ventricles.

The heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage.

The vasculature of the human body, which may be referred to as the circulatory system, cardiovascular system, or vascular system, contains a complex network of blood vessels with various structures and functions and includes various veins (venous system) and arteries (arterial system). Generally, arteries, such as the aorta 16, carry blood away from the heart, whereas veins, such as the inferior 19 and superior 18 venae cavae, carry blood back to the heart.

The aorta 16 is a compliant arterial blood vessel that buffers and conducts pulsatile left ventricular output and contributes the largest component of total compliance of the arterial tree. The aorta 16 includes the ascending aorta 12, which begins at the opening of the aortic valve 7 in the left ventricle of the heart. The ascending aorta 12 and pulmonary trunk 11 twist around each other, causing the aorta 12 to start out posterior to the pulmonary trunk 11, but end by twisting to its right and anterior side. Among the various segments of the aorta 16, the ascending aorta 12 is relatively more frequently affected by aneurysms and dissections, often requiring open heart surgery to be repaired. The transition from ascending aorta 12 to aortic arch 13 is at the pericardial reflection on the aorta. At the root of the ascending aorta 12, the lumen has three small pockets between the cusps of the aortic valve and the wall of the aorta, which are called the aortic sinuses or the sinuses of Valsalva. The left aortic sinus contains the origin of the left coronary artery and the right aortic sinus likewise gives rise to the right coronary artery. Together, these two arteries supply the heart.

As mentioned above, the aorta 16 is coupled to the heart I via the aortic valve 7, which leads into the ascending aorta 12 and gives rise to the innominate artery 27, the left common carotid artery 28, and the left subclavian artery 26 along the aortic arch 13 before continuing as the descending thoracic aorta 14 and further the abdominal aorta 15. References herein to the aorta may be understood to refer to the ascending aorta 12 (also referred to as the “ascending thoracic aorta”), aortic arch 13, descending or thoracic aorta 14 (also referred to as the “descending thoracic aorta”), abdominal aorta 15, or other arterial blood vessel or portion thereof.

Arteries, such as the aorta 16, may utilize blood vessel compliance (e.g., arterial compliance) to store and release energy through the stretching of blood vessel walls. The term “compliance” is used herein according to its broad and ordinary meaning, and may refer to the ability of an arterial blood vessel or prosthetic implant device to distend, expand, stretch, or otherwise deform in a manner as to increase in volume in response to increasing transmural pressure, and/or the tendency of a blood vessel (e.g., artery) or prosthetic implant device, or portion thereof, to recoil toward its original dimensions as transmural pressure decreases.

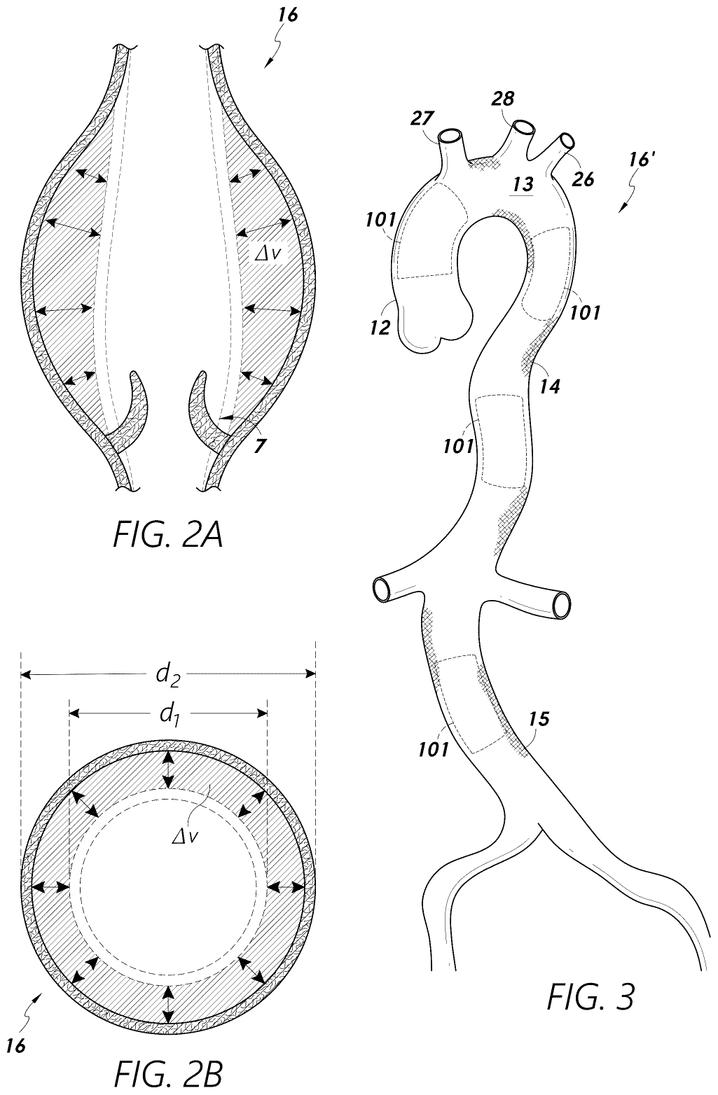

FIGS. 2A and 2B show side and axial cross-sectional views, respectively, of the healthy aorta 16 of FIG. 1 experiencing compliant expansion and contraction over a cardiac cycle.

As referenced above, the systolic phase of the cardiac cycle is associated with the pumping phase of the left ventricle, while the diastolic phase of the cardiac cycle is associated with the resting or filling phase of the left ventricle. As shown in FIGS. 2A and 2B, with proper arterial compliance, an increase in volume Δv will generally occur in an artery when the pressure in the artery is increased from diastole to systole. As blood is pumped into the aorta 16 through the aortic valve 7, the pressure in the aorta increases and the diameter of at least a portion thereof expands. A first portion of the blood entering the aorta 16 during systole may pass through the artery during the systolic phase, while a second portion (e.g., approximately half of the total blood volume) may be stored in the expanded volume Δv caused by compliant stretching of the blood vessel 16 from a non-expanded diameter d₁ to an expanded diameter d₂, thereby storing energy for contributing to perfusion during the diastolic phase. A compliant aorta may generally stretch with each heartbeat, such that the diameter of at least a portion of the aorta expands.

The tendency of the arteries to stretch in response to pressure as a result of arterial compliance may have a significant effect on perfusion and/or blood pressure in some patients. For example, arteries with relatively higher compliance may be conditioned to more easily deform than lower-compliance arteries under the same pressure conditions. Compliance (C) may be calculated using the following equation, where Δv is the change in volume (e.g., in mL) of the blood vessel, and Δp is the pulse pressure from systole to diastole (e.g., in mmHg):

$$C = \frac{Δ v}{Δ p} \qquad ( 1 )$$

In older individuals and patients suffering from heart failure and/or atherosclerosis, compliance of the aorta and other arteries can be diminished to some degree or lost. Such reduction in compliance can reduce the supply of blood to the organs of the body due to the decrease in blood flow during diastole. Among the risks associated with insufficient arterial compliance, a significant risk presented in such patients is a reduction in blood supply to the heart muscle itself. For example, during systole, generally little or no blood may flow in the coronary arteries and into the heart muscle due to the contraction of the heart which holds the heart at relatively high pressures. During diastole, the heart muscle generally relaxes and allows flow into the coronary arteries. Therefore, perfusion of the heart muscle relies on diastolic flow, and therefore on aortic/arterial compliance.

Insufficient perfusion of the heart muscle can lead to and/or be associated with heart failure. Heart failure is a clinical syndrome characterized by certain symptoms, including breathlessness, ankle swelling, fatigue, and others. Heart failure may be accompanied by certain signs, including elevated jugular venous pressure, pulmonary crackles and peripheral edema, for example, which may be caused by structural and/or functional cardiac abnormality. Such conditions can result in reduced cardiac output and/or elevated intra-cardiac pressures at rest or during stress.

FIG. 3 shows an example stiff aorta 16′. As shown in FIG. 3, the aorta tends to change in shape as a function of age, resulting in a higher degree of curvature and/or tortuosity over time. As the vasculature of a subject becomes less elastic, arterial blood pressure (e.g., left-ventricular afterload) becomes more pulsatile, which can have a deleterious effect. For example, undesirably pulsatile arterial blood flow, such as the thickening of the left ventricle muscle and/or diastolic heart failure. Stiffness in the aorta and/or other blood vessel(s) can occur due to an increase in collagen content and/or a corresponding decrease in elastin.

With the walls of the blood vessel 16′ being resistant to stretching due to the stiffness thereof, the expansion of the blood vessel diameter from the non-expanded diameter to the expanded diameter may be limited/reduced compared to the expansion of diameter of a healthy blood vessel. A stiff aorta 16′, as blood pressure increases, may experience a small amount of expansion and volume change, or the blood vessel may be sufficiently stiff that substantially no vessel expansion takes place during systole.

Examples of the present disclosure provide stent implant devices having non-circular frame segments and trapped blood expulsion/aspiration elements, which may be implanted/secured within one or more locations in a compromised aorta and/or other vessel(s). For example, FIG. 3 shows example positions of stents 101 secured to blood vessel walls using features/aspects disclosed herein, such stents being implanted/disposed in various potential areas of the aorta 16′.

### Compliance-Enhancing Stent Implants

Example stent implant devices disclosed herein can be configured to add-back and/or increase compliance in the aorta or other arterial (or venous) blood vessel(s) to provide improved perfusion of the heart muscle and/or other organ(s) of the body. For example, example stent implant devices of the present disclosure can include stents that, when implanted, are configured to decrease the cross-sectional area/volume of a target blood vessel segment in which the stent is implanted during low-pressure conditions, such as diastole, which serves to force blood through the blood vessel segment by pushing the blood through the vessel as the vessel volume reduces in connection with stent contraction induced by cyclical drops in blood pressure.

The non-circular (e.g., oval-and/or peanut-shaped) stents of the present disclosure can advantageously be configured to generate a differential cross-sectional area or volume of the target blood vessel(s) (e.g., aorta) between high-and low-pressure phases of the cardiac cycle to facilitate perfusion. As described above, relatively non-compliant blood vessels generally may not be able to stretch to thereby lengthen the perimeter of the blood vessel in response to increased pressure conditions. Such inability to stretch can prevent compliant expansion of the blood vessel.

Using non-circular stents to produce complaint blood vessel volume change by manipulating/reshaping the native blood vessel walls can increase compliance in a target blood vessel without requiring blood vessel grafting or resection. Therefore, compared to blood flow solutions involving blood vessel grafting/resection, non-circular stent examples of the present disclosure can provide solutions that avoid certain risks that may be associated with cutting of the vessel and/or devices grafted in/to such vessels, which may present risk of rupture and blood leakage outside of the circulatory system.

With respect to a blood vessel having a relatively fixed perimeter, wherein the blood vessel wall does not stretch/expand sufficiently due to stiffness and/or other factors of non-compliance, generally, the greatest area/volume of the blood vessel may be present/achieved when the blood vessel wall forms a circular cross-sectional shape. FIGS. 4-1 and 4-2 show a blood vessel in circular and non-circular axial cross-sectional shapes, respectively.

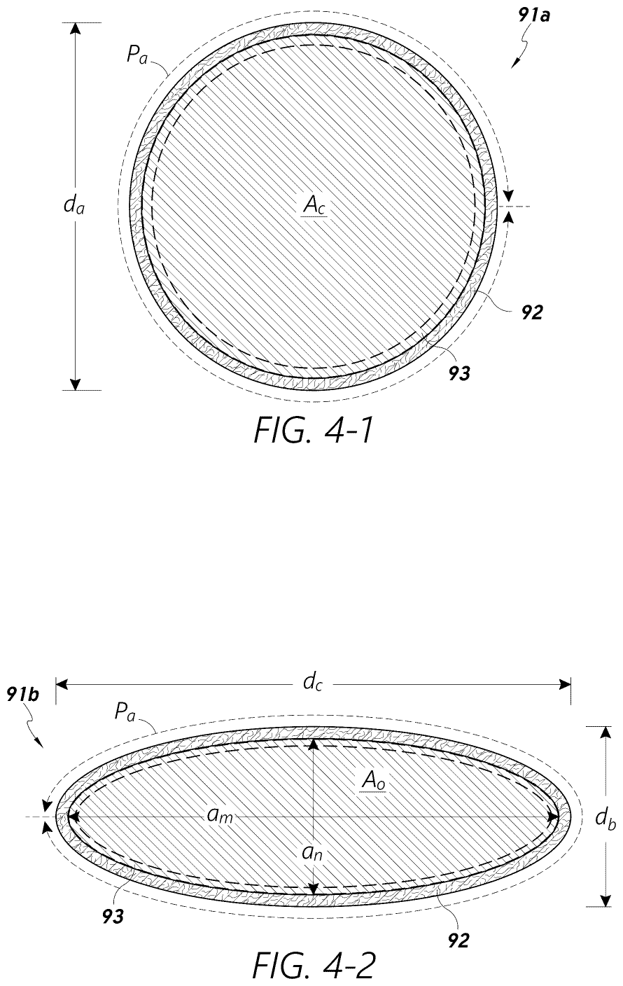

FIG. 4-1 shows an example blood vessel 91 (identified as blood vessel 91a in FIG. 4-1) having a generally circular cross-sectional shape, such that the area A_{c} thereof is maximized for the given perimeter/wall-length Pₒ. In the circular configuration, the diameter dₐ is substantially constant at every angle about the axis of the vessel. The circular shape of the vessel 91a may be set or permitted by the shape of a stent 93 implanted within the vessel.

Diverging from a circular cross-sectional shape can produce a cross-sectional area/volume for a blood vessel that is less than the maximum area A_{c} shown in FIG. 4-1. For example, FIG. 4-2 shows the blood vessel 91 (identified as vessel 91b in FIG. 4-2) having a shape that resembles an oval/ellipse, which produces the cross-sectional area A_{c} that is less than the area A_{c} with the same blood vessel wall/perimeter length Pₐ. The oval shape of the vessel 91b may have a major axis aₘ having a dimension d_{c} that is greater than a dimension d_{b} of the minor axis aₙ thereof. The oval shape of the vessel 91b may be set/forced by the stent 93, which may have a biased oval shape.

With further reference to FIGS. 4-1 and 4-2, due to the area Aₒ of the oval vessel of FIG. 4-1 being less than the area A_{c} of the circular configuration shown in FIG. 4-1, transitioning from the circular shape 91a to the non-circular shape 91b, can provide a reduction in area/volume of the blood vessel, and therefore solutions that cause transitions between circular and non-circular blood vessel shapes between cardiac phases can provide compliance characteristics without the need for elasticity in the blood vessel wall tissue.

In view of the foregoing, examples of the present disclosure provide stent implant devices and associated processes configured to transition the shape/area of a blood vessel from circular/more-circular to non-circular/less-circular shapes, and vice versa, to enhance compliance with respect to the area of the implant reshaping. Such stent implant devices/processes may effect vessel reshaping through dynamic reshaping of the structural shape of the stent in a way that produces a change in shape of the blood vessel in which it is implanted to produce a change in blood vessel area/volume between the systolic and diastolic phases of the cardiac cycle. Trapped blood expulsion/aspiration from around a non-circular stent segment can increase the blood-vessel-reshaping capability of a stent implant, as such trapped blood may otherwise interfere with the re-shaping ability of the stent on the blood vessel. The term “stent” is used herein in accordance with its broad and ordinary meaning and may refer to any device configured to be implanted in a lumen of a blood vessel, the device having a tubular form forming a lumen through which blood can flow.

Examples of the present disclosure provide for stent-type implants that are biased, with respect to at least a lengthwise portion/segment thereof, to a non-circular cross-sectional area, such that, in a relaxed/non-pressurized state, a first diameter of the stent has a greater dimension along a major axis compared to a second diameter of the stent along a minor axis, wherein such stents are configured to transition to a more-circular shape when pressure within the blood vessel overcomes the non-circular bias of the stent and causes the stent walls to be pushed to the more-circular configuration.

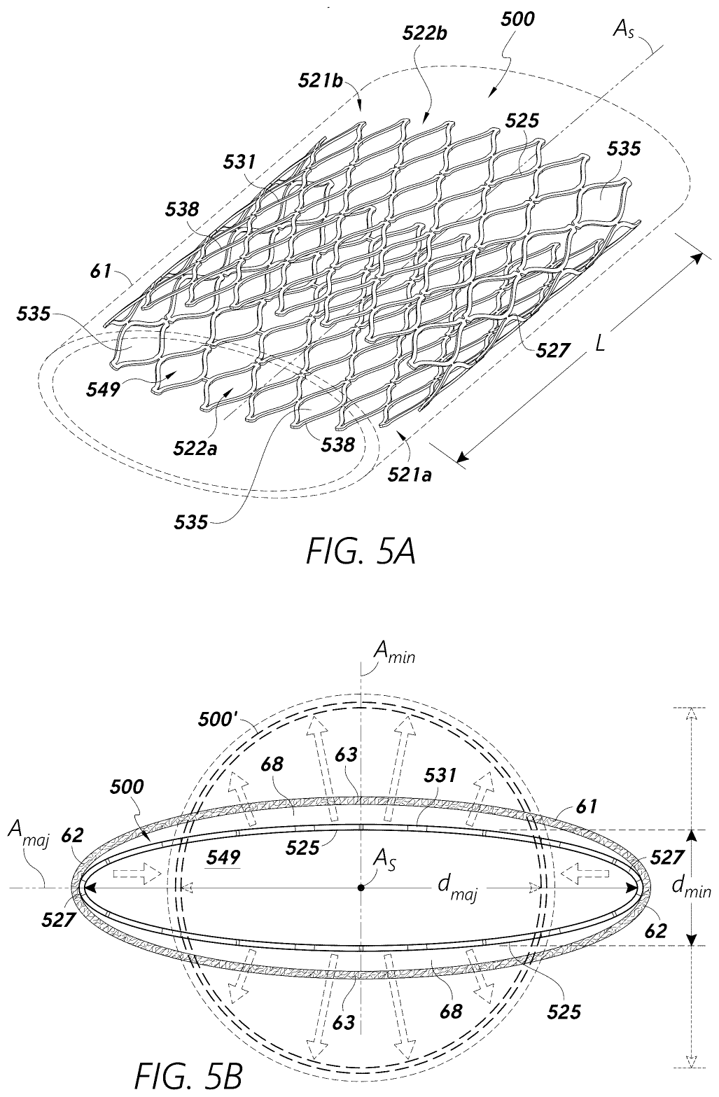

FIGS. 5A and 5B show perspective and axial views, respectively, of a non-circular stent 500 in accordance with one or more examples. Although not shown for clarity in FIGS. 5A and 5B, it should be understood that the stent 500 may comprise one or more trapped blood expulsion/aspiration features/means adapted to facilitate removal of blood from between the stent and the target blood vessel wall. Description of aspects of any example expulsion and/or aspiration element/feature of the present disclosure may be understood to be implementable in example stents like that shown in FIGS. 5A and 5B. The illustrated stent 500 may represent a non-circular segment of a stent implant having one or more circular portions/segments, as described in detail herein.

The stent 500 may be formed of a tubular frame 531, which may form a wall around an axial channel 549, thereby defining the channel 549. As described herein, the frame wall 531 of the stent 500 can be considered a single, circumferentially-wrapped wall, or may be considered to comprise multiple walls, or wall segments. For example, with respect to oval stents and other non-circular stents, as illustrated in FIGS. 5A and 5B, such stents may be considered to comprise sidewall segments 525 that run along relatively long sides of the stent that are aligned generally with the orientation of the major axis/dimension Aₘₐⱼ of the stent, as well as end wall segments 527, which may connect the side walls 525 on major-axis ends of the stent 500. The end walls 527 may be outwardly-curved/concave with respect to an axis Aₛ of the stent 500. The sidewalls 525 may bow/deflect outward, either in a resting, unpressurized state, or in conditions of hoop/wall stress on the frame 531. For example, the sidewalls 525 may bow outward such that the sidewalls 525 are concave from the perspective of the axis Aₛ of the stent 500 and convex from the perspective of the exterior of the stent 500.

Certain stent shapes are described herein, including circular, non-circular-, oval-, peanut-, and other-shaped stents. It should be understood that such description of stent shapes refers to a shape of an axial cross-section of a stent, as depicted in the view of FIG. 5B. Although oval-and peanut-shaped stents are described, it should be understood that the principles of the present disclosure may relate to stents having any non-circular shape in at least some configurations thereof (e.g., in a relaxed/biased configuration). Descriptions of stents in a relaxed or biased configuration should be understood to relate to a configuration that a stent naturally assumes in the absence of tension on the stent wall(s) from external forces (e.g., ambient fluid pressure, physical contact forces, etc.). For example, the biased/relaxed shape of the stent may be due to shape memory of the stent and/or frame thereof.

The stent 500 may be considered an oval stent with respect to the shape of the axial cross-section thereof, as shown in FIG. 5B. The term “oval” is used herein according to its broad and ordinary meaning and may be used substantially interchangeably with the term “ellipse” and/or “oblong,” which terms are likewise used according to their broad and ordinary meanings. The term “oval” may be used to refer to any non-circular closed curve having major and minor axes, the major axis being greater than the minor axis. With respect to “oval”-shaped stents disclosed herein, such stents may have relatively flatter minor-axis sidewalls (compared to curved major-axis end walls), wherein the sidewalls may bow radially outward, and/or may be deflected/curved radially inward so as to produce external concavity and internal convexity in such sidewalls (e.g., forming a peanut-shaped stent). Major-axis walls of an oval stent as described herein may be considered wall portions of a stent that are intersected by a major axis of the stent that runs through an axial center of the stent. Minor-axis walls of such oval stents may be considered wall portions that are intersected by a minor axis of the stent that runs through the axial center of the stent. Example stents of the present disclosure may be considered to have an oval shape whether or not the shape thereof is definable by an algebraic curve. Example stents of the present disclosure may be considered oval stents when the wall(s) of the stent in an axial-cross-sectional perspective form(s) a closed or open curve in a plane that is non-circular; one or more segments/areas thereof may resemble the outline of a portion of an egg. Oval stents of the present disclosure may include either one or two axes of symmetry of an ellipse, such as the illustrated major Aₘₐⱼ and minor Aₘᵢₙ axes. The axial cross-section of some examples of oval stents of the present disclosure may resemble the union of two semicircles on opposite sides of a rectangle, providing a shape evoking the likeness of a speed skating rink or an athletics track. In some contexts, the oval stent 500 may be considered a “stadium”-shaped stent, or an elongated oval.

The stent frame 531 comprises stent wall(s) defining an elongated tubular structure having a first axial end 521a with a first opening 522a. The tubular structure may further comprise a second axial end 521b with a second opening 522b, wherein the lumen/channel 549 extends between the first opening 522a and the second opening 522b, traversing the length L of the stent 500. The frame 531 and/or wall(s) thereof may comprise an open-cell structure adapted to be expanded to secure the stent 500 to a blood vessel internal (or external) wall, such as through endothelialization of the frame 531 to the vessel tissue over time as the frame 531 holds the blood vessel wall using certain tissue-engagement features described in detail herein.

The stent 500 may be elastically deformable between a first, non-circular configuration and a second, more-circular configuration 500′ (see dashed-line representation in FIG. 5B), with the stent 500 biased toward the non-circular configuration. In some examples, the stent frame 531 may comprise a shape-memory material, such as Nitinol. Although shown as an oval-shaped stent, the stent 500 may be any non-circular shape in a relaxed state thereof, such as a triangle, peanut, figure-8, star, clover/lobed, and/or kidney shape.

The stent 500 may be configured to be percutaneously delivered to a blood vessel 61 in a compressed delivery configuration. Once within the blood vessel lumen at the target deployment site, the stent 500 may be configured to be radially expanded into direct surface contact with the blood vessel wall (e.g., the inner wall of an aorta segment). Placement of the stent 500 in the blood vessel may cause at least slight stretching in the blood vessel wall, such as due to pressure at the major-axis ends 527 against the blood vessel wall. The stent wall and/or a portion thereof may be configured to be endothelialized to the blood vessel wall.

In the oval configuration shown in FIGS. 5A and 5B, the stent 500 may have a cross-sectional area having a major/long-axis diameter dₘₐⱼ that is substantially larger than the minor/short-axis diameter dₘᵢₙ. For example, the major-axis diameter/dimension dₘₐⱼ may advantageously be at least twice as long as the minor-axis diameter/dimension dₘᵢₙ, or even 3, 4, 5, 6, or 7 times greater. The stent 500 may be configured to increase compliance of a blood vessel though constant or near-constant pressure at one or more points along a perimeter/circumference of the blood vessel that causes a change in the perimeter geometry of the vessel. For example, the blood vessel may transition from a non-circular/less-circular shape to a circular/more-circular shape.

The stent frame wall(s) 531 may be at least partially composed of struts 538 and/or stent openings/cells 535 between the struts 538. The dimensions and/or shape of the stent 500 may vary based on the particular application and/or target implantation anatomy. For a stent configured for deployment in an aorta, the length L may be between 1-30 cm, and in the biased oval/diastolic configuration the major axis dₘₐⱼ may be between 1-4 cm (or larger/smaller depending on the particular anatomy), and the minor axis dₘᵢₙ can be between 20-50 percent of the major axis dₘₐⱼ. However, other sizes and/or shapes are also within the scope of this disclosure.

The configuration of the stent 500 in the oval shape can cause blood vessel wall 61 to assume a more oval shape to match the shape of the stent 500. However, depending on the relative size of the stent 500 to the vessel 61, the blood vessel 61 may not necessarily conform exactly to the circumference and/or shape of the stent 500, and gap(s) 68 may be present and/or form between the frame 531 and the blood vessel wall 61 as the luminal pressure increases and pushes the vessel side walls away from the frame sidewall 525. Due to the presence of the gaps 68, which may form cyclically as the pressure drops (e.g., during diastole) and the stent transitions to the oval shape, such that the walls 525 pull farther away from the blood vessel wall, desirable sealing between the stent and the blood vessel may be impeded. The presence of the gaps 68 can reduce the ability of the stent 500 to reshape the blood vessel, thereby negatively impacting the efficacy of the stent 500 with respect to compliance-enhancement. Furthermore, blood may collect and/or stagnate to some degree in the gaps 68, resulting in increased risk of embolus/thrombus formation.

When implanted in a blood vessel, the patient physiology may respond to the stent 500 as a foreign object. For example, macrophages can accumulate around the stent, and nearby smooth muscle cells can proliferate to cover the stent. Over time, a new endothelial layer can form over the stent, which can inhibit clot formation. In addition to preventing embolus/thrombus formation, endothelialization can enhance the ability of the stent to reshape the target blood vessel by strengthening the physical coupling between the stent and the blood vessel, thereby reducing the presence of gaps forming between the stent and blood vessel wall when the stent walls pull away from the blood vessel wall as the stent reshapes to an oval/non-circular shape. Tissue overgrowth can be promoted through contact between the stent frame and the blood vessel walls. Examples of the present disclosure provide devices that facilitate contact between non-circular stent frames and blood vessel walls by vacating trapped blood in the gaps 68 and drawing the blood vessel wall 63 into proximity/contact with the stent frame 531, thereby increasing the efficacy of the stents with respect to reshaping/compliance-enhancement.

The stent 500 may be biased toward the illustrated oval and/or other non-circular relaxed/diastolic configuration (shown in solid-line in FIG. 5B), and may, when subjected to mechanical forces associated with high luminal pressure, be configured to responsively transform to a more-circular systolic configuration (shown in dashed-line in FIG. 5B) such that the minor axis dₘᵢₙ approaches, and may equal, the major axis dₘₐⱼ. As with any of the examples disclosed herein, the stent 500 can be configured to deflect from the oval shape to a more-circular shape in the presence of threshold blood pressure levels greater than 80 mmHg, such as blood pressure levels greater than 90 mmHg (e.g., between 90-120 mmHg).

The transition of the stent from oval to the more-circular stent shape 500′ (shown in dashed-line in FIG. 5B) causes energy to be stored in the stent frame 531 (e.g., in the elasticity and/or shape memory thereof), such that energy is returned to the blood vessel walls, and therefore to the blood circulation within the blood vessel segment, when the frame transitions back to the oval shape 500 as pressure decreases.

For examples in which the stent frame 531 is covered internally and/or externally by a fluid-tight covering (not shown in FIGS. 5A and 5B for visual clarity), the openings of the cells 535 of the frame 531 may be closed to pass-through fluid and prevent blood in the blood vessel 61 and within the channel 549 of the stent 500 from transferring pressure through the frame 531. Therefore, intraluminal pressure within the flow channel 549 of the stent 500 loads against the frame 531 (rather than directly against the blood vessel walls 63) to provoke reshaping thereof. The blood-pressure-induced force against the covering and/or stent frame 531 can increase the hoop stress on the frame 531 and/or covering, which may force the frame 531, and with it the blood vessel 61, to assume a more-circular shape. As the pressure in the channel 549 increases (e.g., in connection with the systolic phase of the cardiac cycle), the plastically-deformable nature of the stent frame 531 allows for the sidewalls 525 of the frame 531 to be pushed outward along with the shortening of the stent 500 in the major axis dimension Aₘₐⱼ. When the sidewalls 525 are deflected outward, the channel 549 assumes a more circular cross-sectional shape.

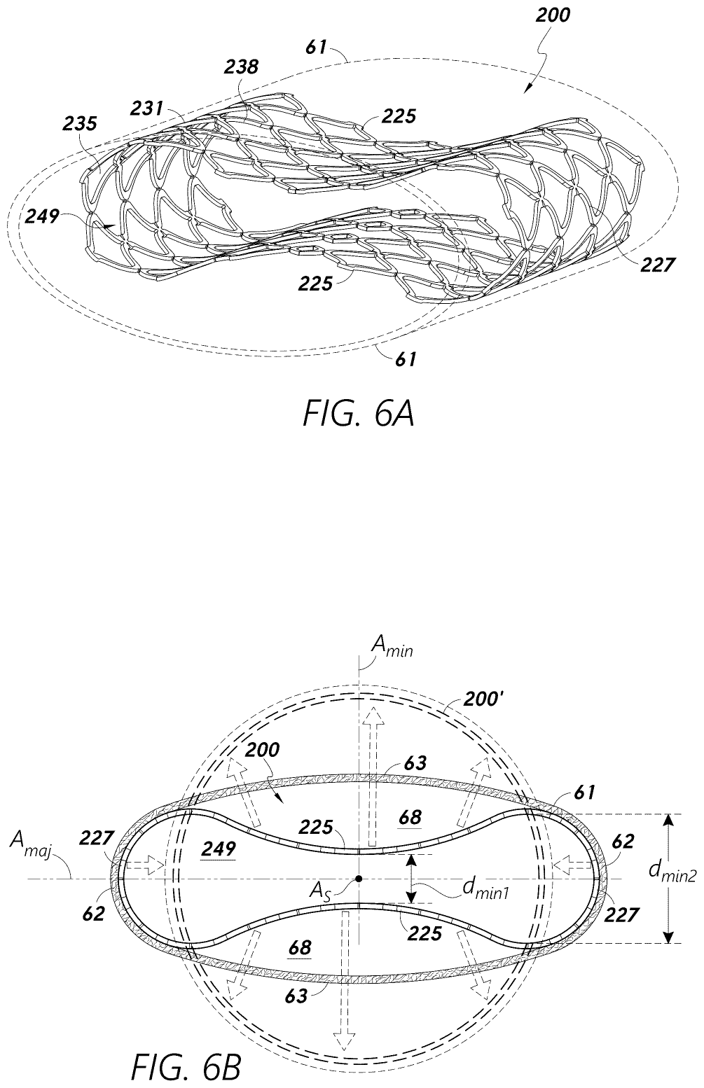

FIGS. 6A and 6B show perspective and axial views, respectively, of a non-circular stent, or stent segment, 200 having inwardly-deflected sidewalls 225 in accordance with one or more examples. The stent 200 may represent an example implementation of any of the non-circular/oval stents/stent-segments disclosed herein, or portion(s) thereof. The shape of the stent 200 deviates from the stent 500 shown in FIGS. 5A and 5B only in that the minor-axis (e.g., relatively flat and/or long) sidewalls 225 deflect to a greater degree towards the center/axis Aₛ of the stent 200 in a relaxed state. The resulting shape may resemble that of an hourglass and/or peanut shape with respect to the axial cross-section shown in FIG. 6B. The stent 200 may have a major axis diameter/dimension that is greater than a minor axis diameter/dimension of the peanut cross-section.

In some examples, the stent 200 may be biased to a shape having a minor axis dimension dₘᵢₙ that is non-constant along the major axis Aₘₐⱼ dimension, which forms an externally-concave/internally-convex surface/form with respect to the minor axis sidewalls 225. For example, the minor axis sidewalls 225 may have a diameter dimension dₘᵢₙ₁ at a center thereof (with respect to the major axis dimension Aₘₐⱼ) that is less than the diameter/dimension dₘᵢₙ₂ at/towards the end portions of the minor-axis sidewalls 225. Non-circular stents of the present disclosure that have externally-concave/internally-convex minor-axis sidewalls as shown in FIG. 6A and 6B are referred to herein as peanut-shaped stents; such peanut shape can be considered a variation of an oval, or oval-shaped, stent as described herein. The inwardly-deflected walls 225 can allow for a transition reshaping between peanut-shaped, to outwardly-bowed oval shape, and ultimately to circular/more-circular shape, shows as the dashed circular stent shape 200′.

Compared to the oval stent 500 of FIGS. 5A and 5B, the peanut-shaped stent 200 may have a tendency to form even greater gaps 68 between the minor-axis sidewalls 225 and the blood vessel walls 61 due to the inward deflection of the walls 225 away from the blood vessel wall. That is, the tendency of the stent sidewalls 225 to pull away from the blood vessel sidewalls 63 may be relatively high, and therefore, without tissue coupling between the vessel walls 63 and the stent walls 225, the reshaping ability/function of the stent 200 may be at least partially impaired/impeded. Furthermore, where the gaps 68 are present between the stent walls 225 and the vessel walls 63, blood may collect and stagnate in such spaces, presenting embolism risk and/or impeding outward deflection of the stent walls 225 to circularize during the systolic phase of the cardiac cycle. Therefore, facilitating tissue overgrowth of the stent walls 225 may be of particular significance with respect to peanut-shaped stents.

In some implementations, stent device of the present disclosure include stent/frame portions that have a circular/cylindrical relaxed shape/form, wherein such stent portions can be integrated with oval portion(s) of the stent in some manner, such that the circular portion(s) of the stent serve to securely hold the stent in place in the blood vessel, whereas the oval portion(s) of the stent can function to increase blood flow (e.g., diastolic flow) through the stent as the oval stent portion transitions between oval and circular/cylindrical configurations in response to changing pressure conditions, as described in detail herein.

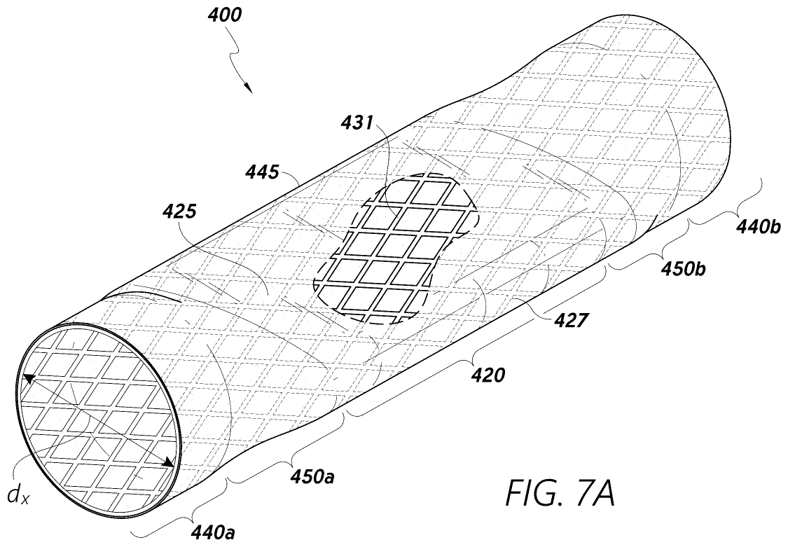

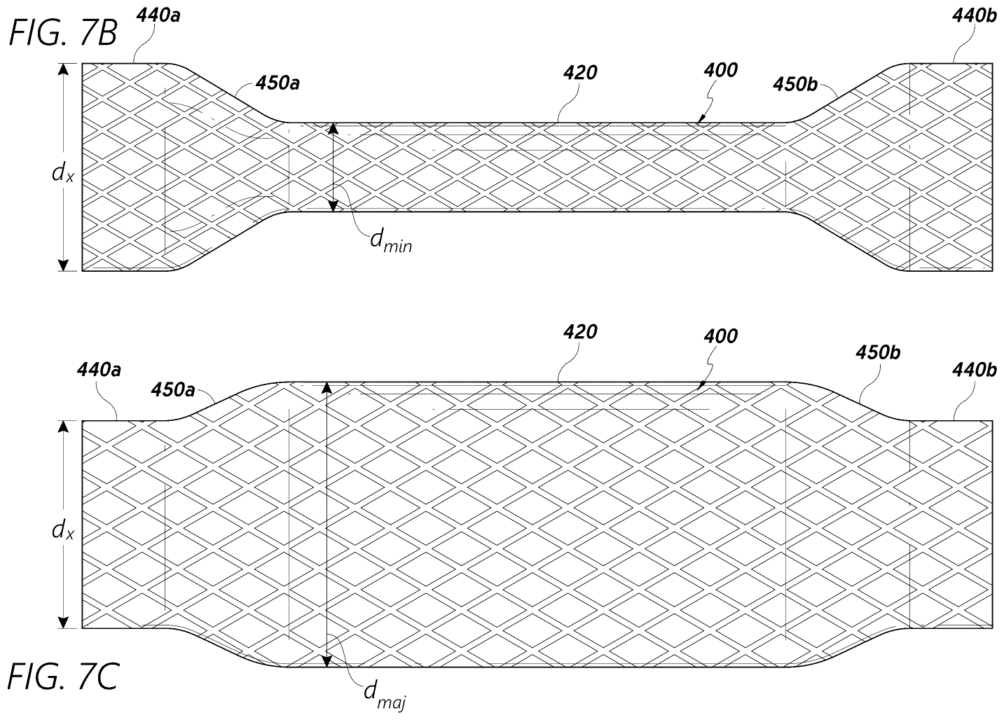

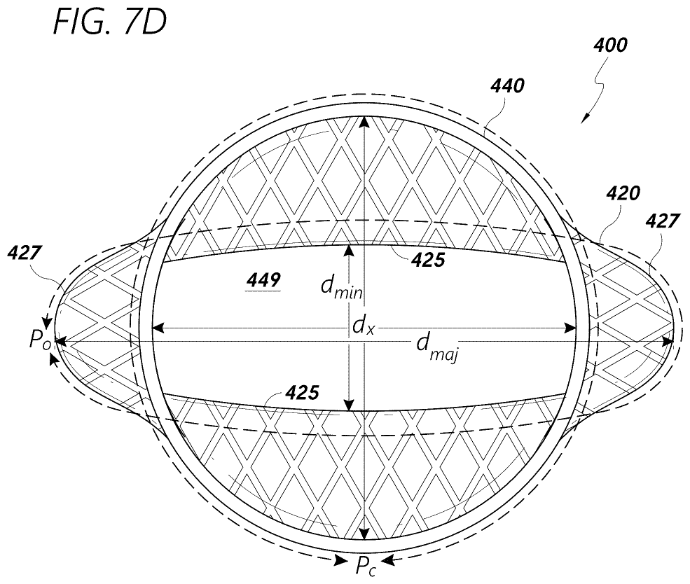

FIGS. 7A-7D show perspective, side, and axial views, respectively, of a stent 400 having circular end portions 440 and a non-circular (e.g., oval) medial portion 420 in accordance with one or more examples. Stent devices having circular axial end portions that provide direct circular contact with the blood vessel at the ends of the stent can particularly benefit from the implementation of trapped blood removal features as disclosed herein with respect to non-circular medial portion(s) thereof. For example, blood collection/stagnation outside such medial stent portions can be reduced and/or eliminated through effective implementation of fluid expulsion features disclosed herein.

As described in detail throughout the present disclosure, oval-shaped and other non-circular stents can be utilized to improve compliance characteristics of a target blood vessel or blood vessel segment. Due to various hemodynamic considerations, as referenced above, it may be desirable to implement such stents in a manner such that a hemostasis seal is present/facilitated on axial ends of the stent. Furthermore, due to the natural circular/cylindrical shape of the blood vessel, oval stents of some examples may not fit as securely within a target blood vessel as compared to traditional circular/cylindrical stents of the same size. For example, while a circular/cylindrical stent may generally distribute contact with the blood vessel wall evenly around the circumference thereof, such that contact between the stent and the blood vessel is not concentrated in specific contact points around the circumference of the stent, oval stents may have a tendency to contact the blood vessel primarily at the major axis ends of the stents oval form, as described in detail above. Furthermore, minor axis sidewalls of oval stent may become spaced/separated from the adjacent blood vessel wall, allowing blood to become trapped between the stent and the vessel wall and impeding blood vessel reshaping. Therefore, oval stents in accordance with aspects of the present disclosure can benefit from blood removal features and processes disclosed herein to prevent/resolve the collection of blood between the stent and the blood vessel wall.

The stent 400 includes circular stent portions 440a, 440b, which are associated with respective axial ends of the stent 400. Although circular portions 440 are shown on both axial ends of the stent 400, it should be understood that circular-oval stent devices of the present disclosure may include only a single circular portion on one end of the device in some cases. The circular stent portions 440 can have any suitable or desirable axial length. In some implementations, the circular portions 440 have a length (in the dimension L) that is less than the oval portion 420. For example, the oval portion 420 may have a length that is at least twice as long as either of the circular portions 440, or twice as long as the combined lengths of the circular portions 440.

The circular portions 440 may be formed of portions of a stent frame 431, which may be integrally formed with the frame portion forming the oval portion 420 of the stent 400. For example, the frame 431 may transition between the circular shape of the circular portions 440 and the oval shape of the oval portion 420. The frame 431 may transition in a relatively smooth/gradual manner from the shape of the oval segment 420 to the circular shape of the circular segments 440 in transition portions/segments 450 of the stent 400. That is, with respect to the flat/long sidewalls 425 that run in the major axis dimension of the oval portion 420, the diameter of the frame 431 may transition from a narrow minor-axis diameter dₘᵢₙ to the circular diameter dₓ of the circular portions 440 moving axially from the oval portion 420 towards the circular end portions 440. Furthermore, with respect to the curved/short end walls 427 of the oval portion 420 that curve around the major axis ends of the oval form thereof, the diameter dₘₐⱼ associated therewith may transition from the relatively long dimension dₘₐⱼ to the relatively shorter circular diameter dₓ moving from the oval portion 420 through the transition portions 450 to the circular portions 440. In some examples, as shown in FIGS. 7A-7D, the diameter dₓ of the circular portions 440 may be less than the major diameter dₘₐⱼ of the oval portion 420, but greater than the minor diameter dₘᵢₙ of the oval portion 420.

As shown in FIG. 7A, the stent 400 may include a covering 445, which is illustrated on an outer surface of the stent 400, although it should be understood that such covering 445 may be disposed within the frame 431 on an inner side/diameter thereof and/or within and without the frame 431, as with other examples of the present disclosure. For clarity, the covering 445 is shown only in FIG. 7A. While a stent frame 431 and covering 445 are shown in FIG. 7A, wherein the covering provides a fluid-tight surface against which pressurized blood within the channel 449 of the stent 400 can press to reshape the oval portion 420 of the stent 400, it should be understood that in some examples, the stent 400 comprises a planar/sheet form that is fluid tight in one or more portions thereof, wherein such planar/sheet form provides the structure/frame of the stent 400 and substitutes for the illustrated strut-based frame 431 and covering 445.

The stent 400 is formed with the central/medial oval-shaped segment 420, which assumes and oval or other non-circular shape at least in a free/relaxed state thereof, with both ends of the stent having circularly-shaped forms so as to fully-engage with the surrounding vascular wall. In some implementations, the end portions 440 can be relatively oversized to conform with and press against the blood vessel walls, which can improve hemostasis, thereby restricting blood flow to solely through the stent's lumen 449. The oversizing of the circular portions 440 relative to the native blood vessel can further help the stent 400 resist migration in either axial direction.

The stent 400 and/or frame 431 thereof may have the same perimeter length P_{c} in the circular portions 440 as in the oval portion 420 (perimeter Pₒ shown in FIG. 7D). In some implementations, the perimeter Pₒ of the circular portions 440 is greater than the perimeter P_{c} of the non-circular portion 420. The transition segment/portions 450 may likewise have the same perimeter as the end 440 and oval 420 segments. The perimeter P_{c} of the circular portions 440 may be selected to match the circumference of the target blood vessel segment. For example, the perimeter P_{c} may be within 10% of the length of the circumference of the target blood vessel in the implantation area. The circumference/perimeter P_{c} of the stent may be slightly larger than that of the target blood vessel prior to deployment of the stent 400, to thereby promote secure attachment of the stent 400 to the blood vessel.

When the stent 400 is implemented with a covering 445, the circular end-portions 440 can serve as funnels that restrict blood flow to flowing through the channel of the medial portion 420, and not flowing around the outside of the medial portion 420. However, as the circular end portions 440 may not be configured to change in shape throughout the cardiac cycle as pressure changes, such portions may not contribute to the stent's compliance-enhancing, vessel-reshaping effect. Moreover, the circular shape of the end portions 440 may cause the end portions to serve as stationary harnessing portions that may limit the movement of the central/medial 420 portion between systolic and diastolic phases.

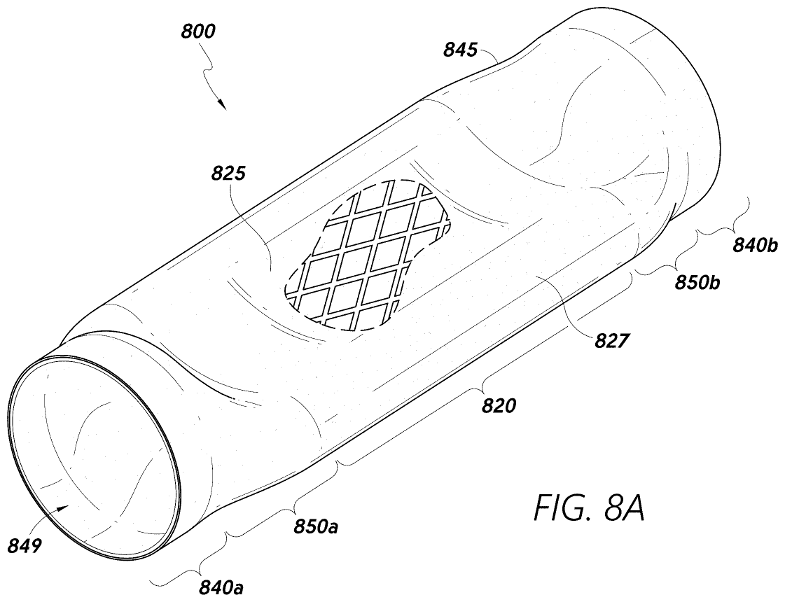

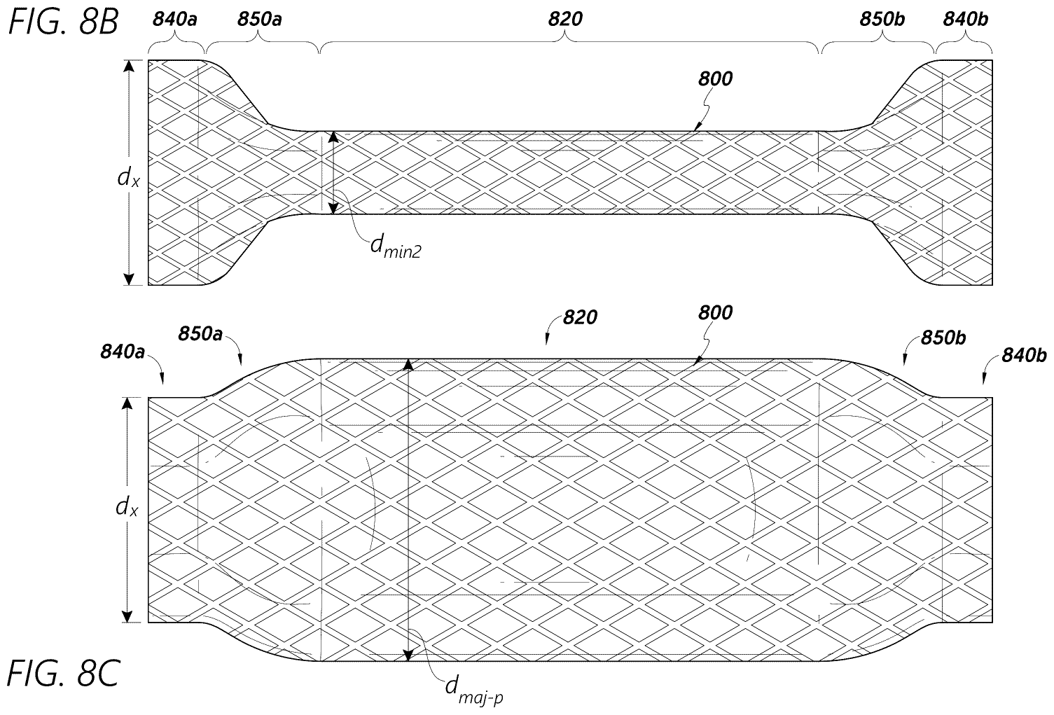

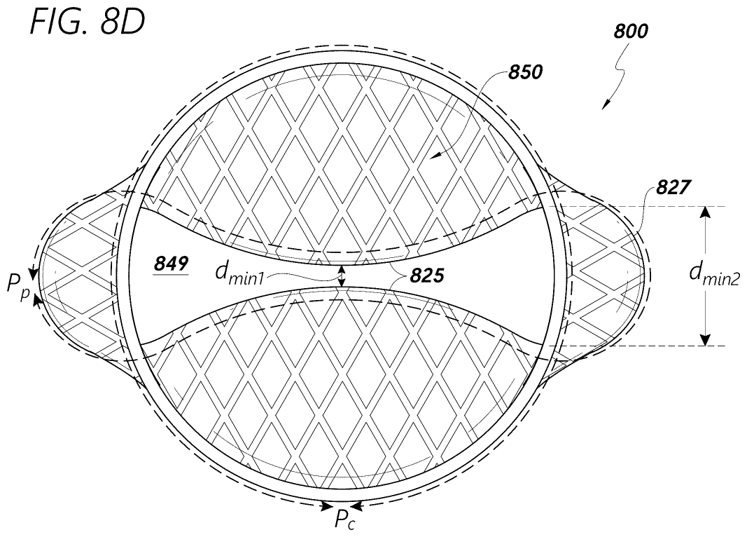

Trapped blood removal can be of particular benefit for stents including peanut-shaped stent segments, due to the potentially greater gap volume between the peanut-shaped stent segment and the blood vessel wall in which blood can become trapped. FIGS. 8A-8D show perspective, side, and axial views, respectively, of a stent 800 having circular end portions 840 and a non-circular (e.g., peanut-shaped) medial portion 820 in accordance with one or more examples.

The stent 800 may have any of the features disclosed above in connection with the stent 400 of FIGS. 7A-7D. However, with respect to the medial portion 820 of the stent 800, such portion may have a cross-sectional axial shape resembling the peanut-type shape disclosed in FIGS. 6A and 6B. Similarly to the stent 400 of FIGS. 7A-7D, the stent 800 can include circular axial ends 840 and transition portions/segments 850 that transition the shape of the stent 800 between the circular ends 840 and the peanut-shaped medial portion 820.

The medial portion 820 may have any suitable or desirable length, and may comprise inwardly-deflected minor axis walls 825, which are connected at major axis ends 827 as with other examples disclosed herein. The medial portion 820 may have any suitable minimum diameter dₘᵢₙ₁ (see FIG. 8D) and/or any suitable or desirable maximum minor-axis dimension dₘᵢₙ₂. Furthermore, the curvature and/or angle(s) of deflection of the walls 825/827 of the peanut-shaped medial portion 820 may have any suitable or desirable parameters. The stent frame 800 can comprise a solid-surface form, rather than a structure formed of struts and open cells as illustrated and described in detail herein.

From the top view of FIG. 8C, it can be seen that in some examples, the

peanut-shaped medial portion 820 has a major diameter dₘₐⱼ₋ₚ that may be greater than a diameter dₓ of the circular portion 840. Conversely, the minor axis dimension dₘᵢₙ₂, as shown in the side view of FIG. 8B, may have a value less than the diameter dₓ of the circular portions 840. In some examples, the perimeter/circumference P_{c} of the circular portions 840 may be substantially equal to the perimeter/circumference Pₚ of the peanut-shaped portion 820 and/or at least a portion thereof. Therefore, when the peanut-shaped portion 820 transitions to a more-circular shape, the cross-sectional shape thereof may resemble that of the circular ends 840.

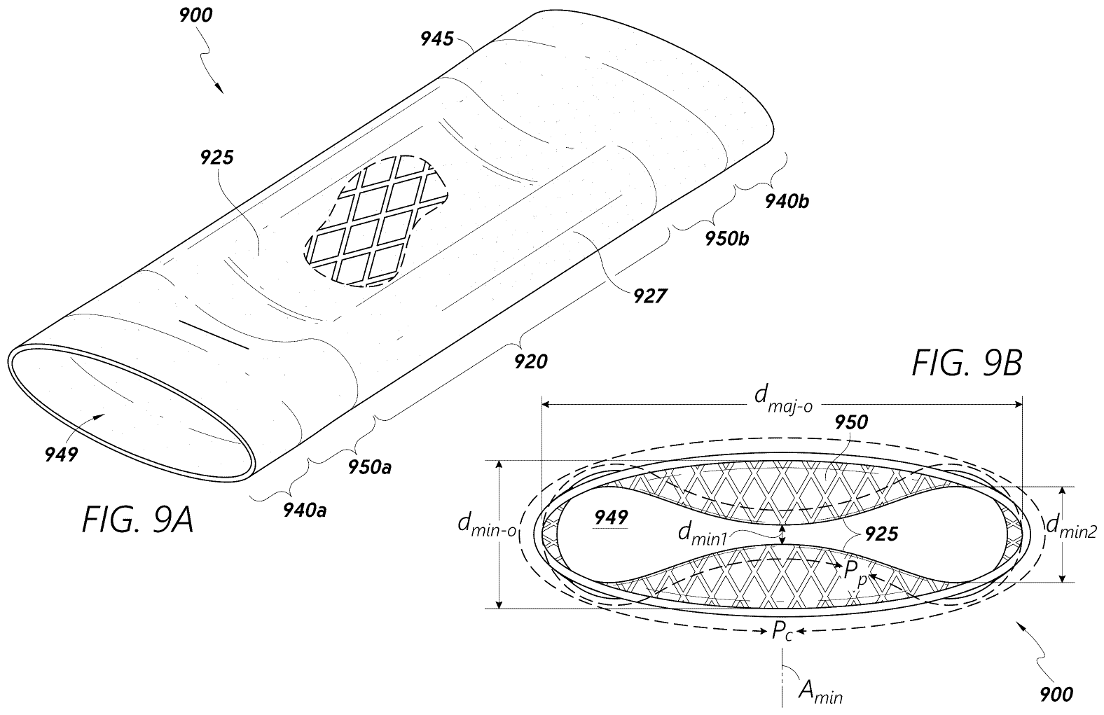

In some implementations, the present disclosure relates to variations of stent designs having differently-shaped end and medial portions/segments. For example, aspects of the present disclosure can provide stents having end portions that are not circular, but rather some non-circular shape that is different than the non-circular shape (e.g., peanut shape) of the medial portion of the stent. In such examples, one or both ends of a compliance-enhancing stent can be designed, with respect to a relaxed shape/configuration thereof, as compliant oval-shaped portions, which may advantageously retain proper sealing against the blood vessel, while also contributing to the stent's blood-vessel-reshaping functioning during transitions between low (e.g., diastole) and high (e.g., systole) pressure phases/conditions. Such stent end shapes can further form more gradual transitions in shape that reduce the likelihood of flow disturbances through the stent. FIGS. 9A and 9B show a stent 900 having oval end portions 940 with radially-outwardly-deflecting/bowing minor-axis sidewalls and an oval medial portion 920 with radially-inwardly-deflecting/concave minor-axis sidewalls (e.g., peanut shape), in accordance with one or more examples.

The stent shape of the stent 900 of FIGS. 9A and 9B may have certain advantages over other stent designs with a peanut-shaped medial portion and including non-compliant circular end portions. For example, while for some designs with circular end portions. such end portions may not be configured to change in shape throughout the cardiac cycle, and thus may not contribute to the stent's compliance-enhancing vessel-reshaping functionality, the oval end portions 940 of the stent 900 may be configured to change in shape in a cyclical manner, thereby contributing to compliance enhancement. Furthermore, while circle-shaped end portions may limit the transitional movement of a stent's central/medial portion, the oval-shaped end portions 940 may be less limiting with respect to the transitional movement of the medial, peanut-shaped portion 920 between the relaxed peanut shape thereof and a more-circular configuration. In addition, while some designs with circular end portions may present relatively abrupt transitions from the circular shaped ends to the narrower (e.g., significantly narrower) peanut-shape of the medial portion of the stent, which can result in flow disturbances, and may present manufacturing complication associated with significant deformations/strains, the transition between the oval shape of the end portions 940 and the peanut shape of the medial portion 920 may have less of an effect on the flow and structural design strains/deformations than some circle-end stents.

The stent design of FIGS. 9A and 9B, when implanted in a blood vessel, may be prone to the collection of blood in the medial portion 920 in the space formed by the inward deflection of the minor-axis walls 925. Therefore, fluid removal processes and features as disclosed herein may advantageously be implemented to expel and block blood from the space between the stent frame and the blood vessel. Furthermore, advantageously, the oval shape of the end portions 940 can be configured/dimensioned to retain contact along its entire circumference with the blood vessel (as with circular end portions of examples disclosed herein), thus preserving proper sealing against the artery wall and channeling the flow into the stent's lumen through the medial portion 92.

As described above, various solutions of the present disclosure using stents having non-circular segments can be implemented for treating hypertensive patients, such as heart failure patients, by improving aortic compliance through blood vessel reshaping. Such stent devices, as described above, can include circular end segments, which may facilitate sealing of the stent implant between the stent and the outer blood vessel wall. For example, stent implants in accordance with aspects of the present disclosure can be configured as a circle-oval/peanut-circle implants, examples of which are shown in FIGS. 7A-7D and 8A-8D, or oval-peanut-oval shaped stents, as shown in FIGS. 9A and 9B.

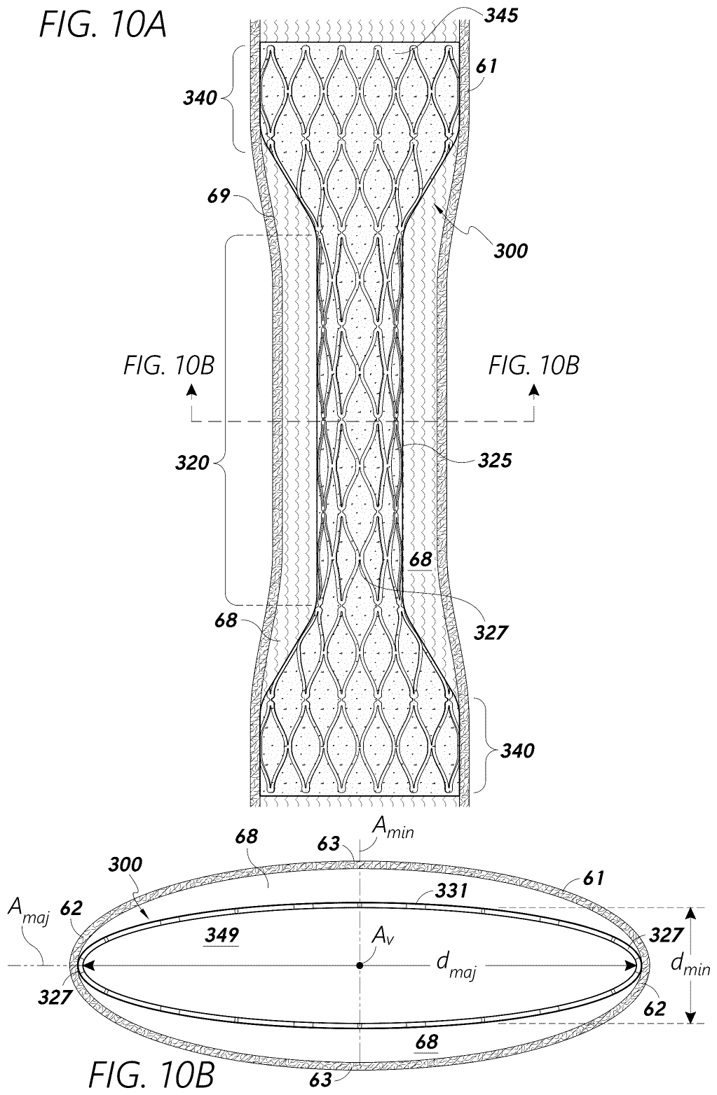

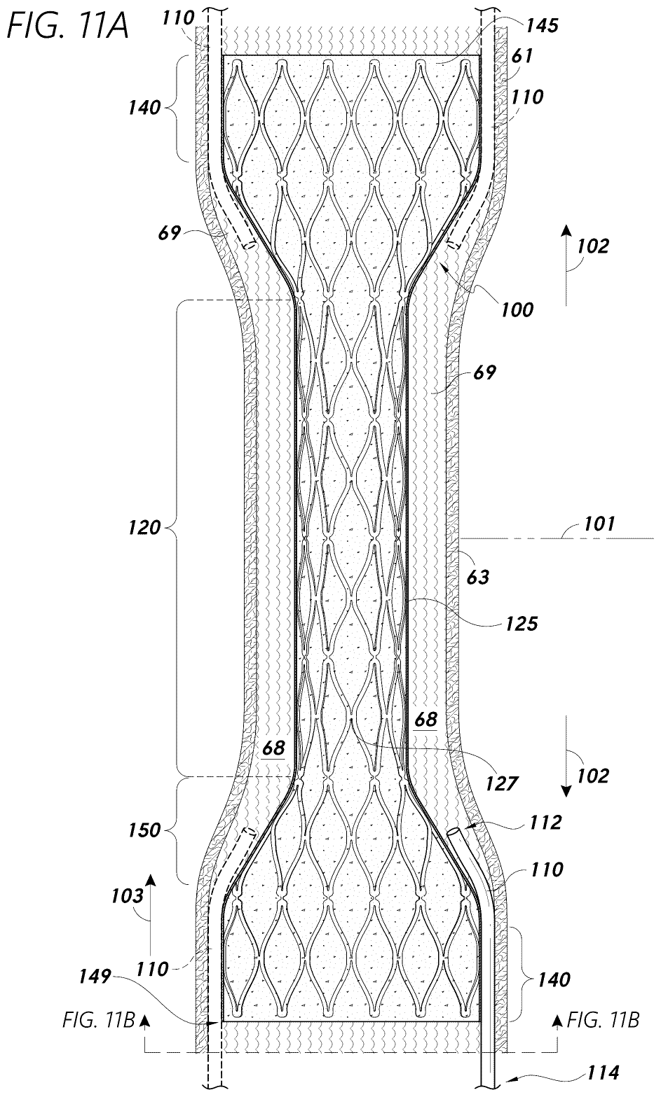

As non-circular stent implants having circular sealing end portions may generally include non-circular medial portion(s)/segment(s) that have a minor axis that is less/shorter than the minor axis of the end segments of the implant, certain volume(s) of blood can become trapped between the stent implant's outer (and/or inner) cover and the blood vessel wall outside of the implant in the blood vessel segment spanned by the medial segment of the stent implant. For example, blood may become trapped between the stent's cover along its non-circular/oval portion between the end segments of the stent and the blood vessel's inner wall. FIGS. 10A and 10B show side and axial views, respectively, of a stent implant device 300 implanted within a blood vessel 61 with trapped blood 69 collected around the stent implant 300 in accordance with one or more examples.

Stagnated blood present in the gaps 68 between the non-circular stent segment 320 and the blood vessel wall 61 can present certain issues. For example, stagnated blood can pose a risk of thrombus formation in the gap area(s) 68, wherein such thrombi formed on the stent implant can compromise the re-shaping functionality thereof. Furthermore, thrombus formation can result in myocardial infarction and/or stroke if dislodged from a position within the gap areas 68 and passing into the bloodstream. Therefore, it may be desirable to implement blood-vessel-reshaping stent implant devices of the present disclosure in a manner as to reduce the presence/occurrence of trapped blood 69 between covered non-circular stent segments and the surrounding vascular anatomy 61, thereby reducing the risk of thrombus and/or otherwise improving the efficacy/functionality of the implant device. Over time, stagnated blood 69 in the gap areas 68 can become solidified and/or tissue-like in a manner that effectively thickens the blood vessel wall 61. Stagnated blood in the gap areas 68 can further defeat the blood-vessel-reshaping effect of the stent due to the generally non-compressible blood 69 occupying the space 68 around the stent frame 331 creating obstruction with regard to the ability of the stent frame to become circularized as pressure levels increase. For example, as the blood vessel segment 61 may have compromised compliance characteristics, the ability of the blood vessel to stretch to accommodate both the expanded circularization of the stent frame 320 and the blood 69 trapped outside of the stent frame may be reduced, and therefore the volume change produced through stent reshaping during cardiac cycling may be limited/reduced, along with the compliance effect thereof.

### Stent Gap Fluid Removal

As discussed above, covered stents having oval-shaped segments in a free state thereof can be implanted inside a blood vessel (e.g., aorta) so as to force the blood vessel to assume a more oval shape under low pressures (e.g., diastole), wherein increased blood pressure (e.g., during systole) forces the stent (and with it the vascular segment/section) to assume a more circular shape, so as to restore some compliance to the otherwise non-compliant (or less compliant) section. Among risks associated with implantation of certain such covered stent implants, blood can become trapped between the outer (or inner) cover of the oval-shaped segment of the stent and the vascular wall upon deployment when the non-circular stent segment is positioned between two circularly-shaped ends of the stent implant, which may be implemented to provide scaling around the stent and/or to prevent unintentional axial migration of the stent after deployment. Blood stagnation in these regions/spaces can result in thrombosis and/or other complications. Thrombus formation in areas/spaces around the outer diameter of the non-circular/oval segment of a stent implant device can press against the stent and thereby compromise the vessel-reshaping functionality of the stent. Furthermore, thrombus can result in myocardial infarction or stroke if dislodged and escaping from the trapped space/position into the blood stream. Various solutions are presented herein to remove trapped blood from around a non-circular stent segment through various blood-flow passages/features using active aspiration/suction or pushing/expulsion of the blood from the trapped space.

### Gap Fluid Removal Using Tubes

According to aspects of the present disclosure, example stent implant devices can be implemented with blood-flow passages/tubes, wherein suction may be applied to such tubes/passages to aspirate trapped blood from a gap area around a non-circular stent segment. Additionally or alternatively, removal of blood through tube/passage features can be through passive or expulsion flow. Example stent implant devices can include fluid passage/tube features that may be utilized for trapped blood removal during and/or after stent deployment.

Trapped blood/fluid removal can cause the blood vessel walls to draw sufficiently close to the stent implant (e.g., cover and/or frame thereof) to prevent or reduce the presence of gap space(s) between the stent and the blood vessel wall, thereby reducing or eliminating the available space for trapped/stagnated blood to collect outside of the stent frame. With the blood vessel wall drawn closely to the stent structure, endothelialization of the stent walls to the blood vessel walls may advantageously occur over time, which can provide for improved blood-vessel-to-stent coupling, which facilitates blood vessel re-shaping. For example, when a blood vessel is reshaped exclusively through major-axis pushing on the blood vessel, a relatively large force may be required to reshape the blood vessel to the desired oval shape. However, where minor-axis attachment/coupling between the stent and the blood vessel occurs in a manner as to provide for pulling on the tissue wall by the minor-axis stent walls, relatively less major-axis pushing/stretching may be required to achieve comparable blood vessel volume change/reshaping due to the ability to collapse the minor-axis blood vessel walls radially inwardly from the inside using the stent frame. Trapped blood may be aspirated or expelled through any type of egress channel, such as a tube and/or one-way valve.

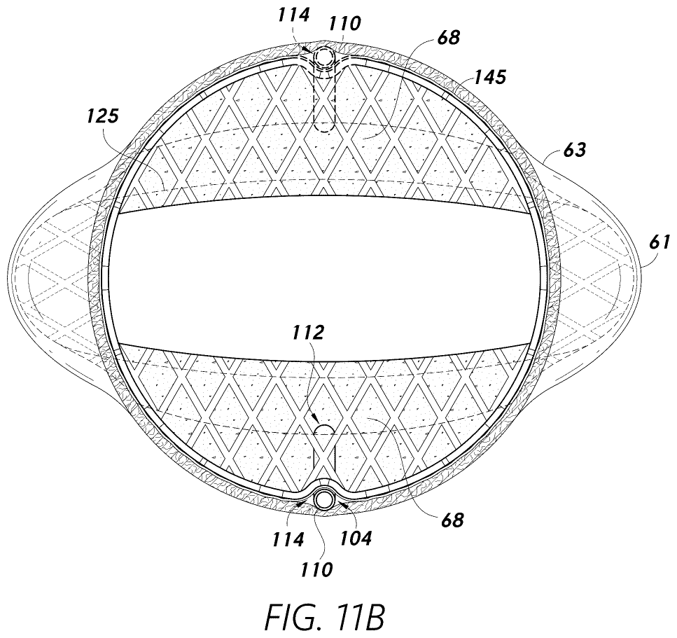

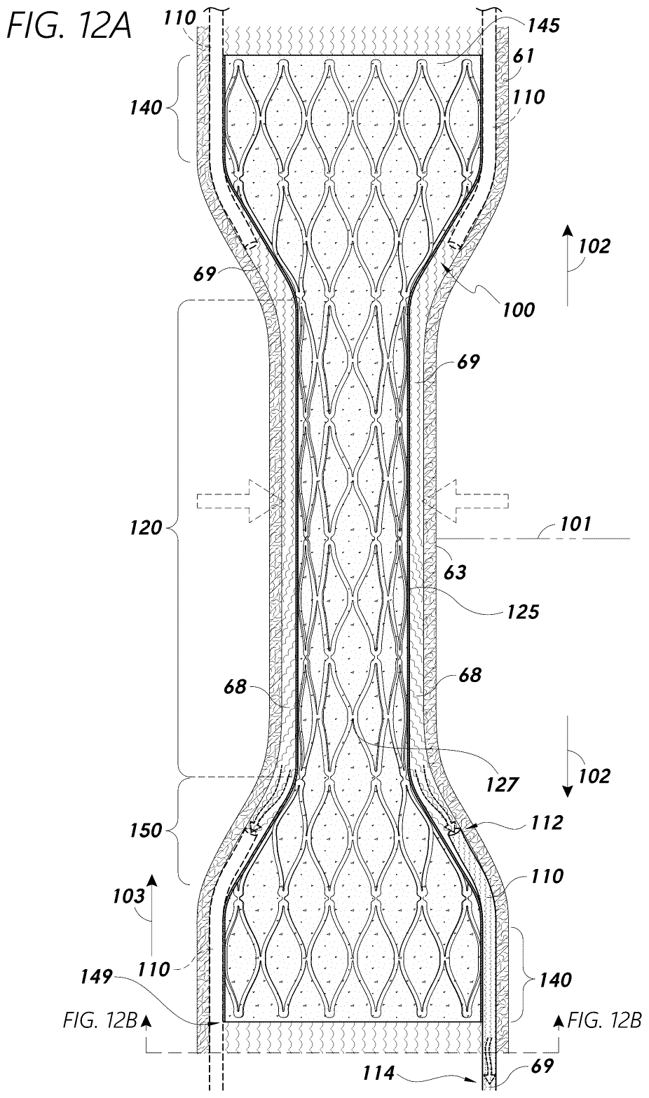

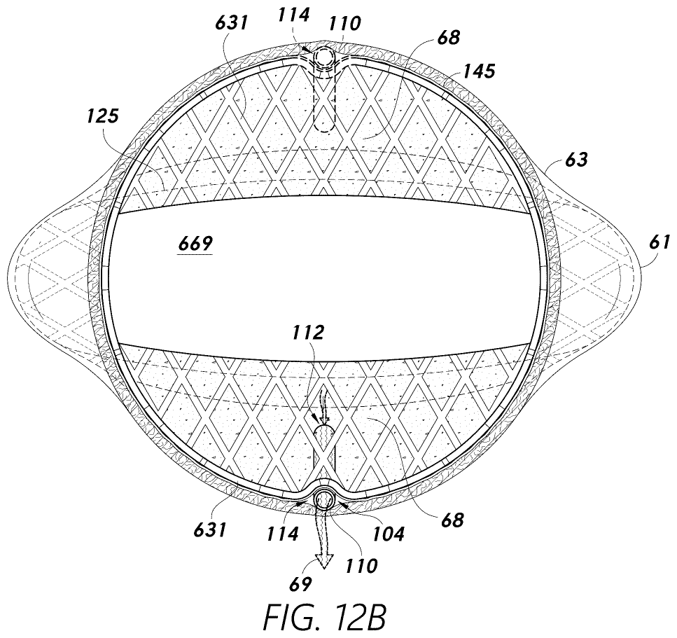

FIGS. 11A and 11B show side and axial views, respectively, of a stent implant device 100 including fluid aspiration/expulsion tube(s) 110 in accordance with one or more examples. FIGS. 12A and 12B show side and axial views, respectively, of the stent implant device 100 having blood passed through the fluid aspiration/expulsion tube(s) 110 associated therewith, which provide egress channel(s) for trapped blood. The illustrations of FIGS. 11A and 12A shows the minor-axis side view of the stent implant 100 to relatively clearly illustrate the gap areas 68 between the minor-axis walls/sides 125 of the non-circular stent frame portion 120 and the blood vessel wall 63 in such areas. The gap area(s) 68 may be considered space on or outside of the outer diameter of the stent segment 120. Removal of the blood 69 through the egress channels/tubes 110 can be via active suction/aspiration or through expulsion through expansion of minor-axis walls 125 of the stent 100.

The implant 100 includes one or more suction/passage tubes 110, which may be component(s) of the delivery system used in connection with the implant 100. Alternatively, the tube(s) 110 can be separate from the delivery system. For example, the tube(s) 110 can be positioned between the stent 100 and the vascular wall 61 prior to, or during, stent deployment. The stent implant 100 includes a cover 145, which may be fluid/liquid-tight. Although described as ‘fluid-tight,’ cover features disclosed herein may not be fully fluid-blocking in some implementations, but may be at least partially impermeable, non-porous, and/or impeding or obstructing of at least certain types of fluid therethrough.

The inner opening/end 112 of the tube(s) 110 can be positioned in fluid communication with the blood volume trapped between the medial segment 120 of the stent 100 and the vascular wall 63, such that application of vacuum force (or other negative pressure) through the tube(s) 110 can serve to draw the trapped blood 69. In some implementations, suction can be applied to the tube(s) 110 once the stent 100 is fully deployed, in a manner such that the trapped blood region 68 is sealed and exposed only to the tube opening(s) 112. Suction of the blood 69 through the tube(s) 110 can result in the blood vessel walls 63 being drawn closer to the stent, such as up to full contact with the outer surface of the stent).

The tube(s) 110 can have any suitable or desirable shape and/or configuration. In some examples, the implant 100 is implemented with a single suction/outflow tube 110. Alternatively, a plurality of tubes can be disposed around the circumference of the stent 100. In some examples, a ring-shaped tube may be implemented that surrounds at least a portion of the stent 100.

When suction/removal of blood 69 from the area 68 is completed, the tube(s) 110 can be optionally withdrawn from the patient's body, leaving the stent 100 implanted without the tube(s) 110 in-place around the stent 100. After suction/removal of the fluid 69, the blood vessel wall 63 may be disposed relatively tightly and/or conforming around the stent body. In some implementations, the tube(s) 110 can remain in position around the stent 100. For example, the after suction/removal of the trapped blood 69 (or at least a portion thereof), the tube(s) 110 may be disconnected from the delivery system and sealed. Sealing of the tube(s) 110 may be accomplished using unidirectional valves associated with the flow channel of each of the tube(s) 110, wherein such valves may be configured to allow flow to pass in the direction 102 away from the axial center 101 of the stent 100, while blocking or impeding flow towards the axial center 101 (e.g., towards the gap area 68).

The tube(s) 110 may be positioned on an outer diameter of circular end portions 140 of the stent 100. For example, the tube(s) 110 may be disposed on an outer diameter of at least a portion of the stent frame and/or cover of the implant 100. The tube(s) 110 may be integrated with the frame, covering, and/or other feature or aspect of the stent implant 100, or may be separate components positioned/disposed on the outside diameter of the, e.g., circular portion 340 of one or more ends of the implant. In some implementations, tube(s) 110 may be associated with only one axial side of the implant 100, such as a proximal side with respect to a delivery system used to deploy the implant 100 and/or implement suction through the tube(s) 110. For example, the blood 69 may be aspirated through one axial side of the implant 100, such side being the side from which the delivery system or other instrumentation deploys the implant 100 and/or aspirates/sucks the blood 69.

The tube(s) 110 may be attached or secured in some manner to the stent. 100, such as through suturing, clamping, adhesive coupling, or other attachment means or mechanism, which may facilitate secure attachment to the stent body/frame and/or prevent dislodging and/or migration of the tube(s) 110 after deployment thereof. In some implementations, the tube(s) 110 is/are held in place through a frictional compression between the stent 100 and the blood vessel wall 61.

The stent implant 100 may be deployed with the tube(s) 110 positioned in contact therewith, or the tube(s) 110 and stent 100 may be deployed separately. For example, in some implementations, one or tubes may be deployed within the blood vessel 61, wherein the stent/frame 100 may be subsequently expanded within the blood vessel 61 in a position such that the tube(s) 110 extend from beyond an axial end 149 of the stent frame 100 and inward 103 into the gap area 68 on the outside of the minor-axis walls/segment 120 of the stent 100 in the medial portion thereof.

The tube(s) 110 may be utilized to remove fluid from the gap areas 68 to improve the functionality of the blood vessel reshaping of the stent implant device 100 in the non-circular medial portion 320 thereof. The removal of blood through the tube(s) 110 can be performed in any suitable or desirable way, such as through active expulsion and/or aspiration of the blood 69 through the tube(s) 110. For example, with respect to aspiration, section force may be applied to the tube(s) 110 to suck the blood 69 in the gap portion 68 through the tube(s), thereby causing the blood vessel walls 63 to be drawn closer to the stent frame, as shown in FIGS. 12A and 12B. Suction/aspiration through the tube(s) 110 can be achieved using a separate instrument/system from the delivery system used to deploy the stent implant device 100 and/or tube(s) 110, or the delivery system used for stent deployment may include functionality for aspirating fluid. Aspiration may be performed after deployment of the stent implant device 100, wherein the collected blood 69 that becomes trapped in such process may be drawn through the tube(s) 110 after deployment of the stent implant device 100 to decrease the gap 68 between the stent frame and the blood vessel wall 61 at least in the area of the medial portion 120.

In some implementations, blood 69 may be passed through the tube(s) 110 through expulsion by expanding and/or reshaping the stent frame in at least the medial portion 120. For example, after deployment of the stent implant device 100, and/or during a deployment process, the minor-axis walls 125 of the stent frame may be expanded from there biased reduced diameter to cause the stent frame to circularize and/or fill the space 68 between the stent 100 and blood vessel wall 61, thereby reducing the available gap space 68 on the outer diameter of the stent 100 and forcing the blood 69 occupying such space out through a path of least resistance, such as through the tube(s) 110.

In some implementations, the tube(s) 110 may comprise one-way valves configured to permit blood/fluid to pass out of the gap areas 68 through the tube(s) 110, while preventing blood from reentering/entering the gap areas through the tube(s) 110.

The process of removing the blood 69 from the gap area 68, as shown in FIGS. 12A and 12B, can be performed to draw the blood vessel walls 63 to the stent body 100 to improve the re-shaping ability of the stent 100 with respect to the blood vessel 61 around the medial segment 120. After removal of the gap fluid/blood 69, the tube(s) 110 may be maintained in position on the outer diameter of the stent 100, or may be removed. In implementations in which the tube(s) 110 is/are removed, the stent 100 may provide a seal in the circular end portions 140 thereof after removal of the tube(s) 110. For example, the presence of the tube(s) may cause radial inward deflection 104 of the stent frame 100, as shown in FIG. 11B, wherein shape memory/configuration of the circular portion(s) 140 of the stent 100 may cause such portions to expand to a more circular/continuous expanded form after removal of the tube(s) 110. Alternatively, the presence of the tube(s) 110 may cause outward deflection of the blood vessel wall in the areas of the tube(s), wherein removal of the tube(s) 110 may allow for the blood vessel 61 to assume a more continuous circular shape in such areas.

The tube(s) 110 may have any suitable or desirable shape and/or length. For example, although shown as having a circular cross-sectional area, it should be understood that the tube(s) 110 may have any suitable or desirable cross-sectional area, such as an oval, rectangle, diamond, nor other shape. Furthermore, the length of the tube may extend any distance past the axial ends 149 of the stent 100 and/or into the gap area 68. In some implantations, the tube(s) 110 may deflect radially inwardly in the transition areas 150 of the stent 100, as illustrated, to more closely conform to the shape of the stent frame in the non-circular segment 120 thereof and/or transition segment. That is, the axially-inner end of the tube(s) 110 may be deflected radially inwardly, as shown. An aspiration pump or similar device may be used to draw the trapped blood 69 through the tube(s) 110.

The tube(s) 110 may extend into a delivery system or other instrumentation configured to implement suction/aspiration through the tube(s) 110. In such examples, removal and/or withdrawal of the suction system/instrumentation may involve pulling the tube(s) 110 from around the stent segments 140 and out of the patient. In some implementations, the tube(s) 110 are cut-off at a position within the blood vessel outside/past the axial end 149 of the stent. In such implementations, the tube(s) 110 may remain within the blood vessel with the stent 100 post-implantation. Suction/aspiration may be implemented using any suction/vacuum device know to those having ordinary skill in the relevant art.

### Gap Fluid Expulsion Using Unidirectional Valves

Examples of the present disclosure provide stent implant devices including certain one-way valve features for removal of blood trapped radially outside of a non-circular segment of a covered stent implant device, wherein the valve features can reduce the risk of thrombus formation due to blood stagnation in regions around the stent segment. Blood-removal valve features of the present disclosure can comprise unidirectional valves disposed around at least a portion of an end segment (e.g., circular end segment) configured allow blood flow from the trapped volume to the blood stream, wherein such expulsion of blood may occur as the non-circular stent segment transitions to a more circular shape during systole, thereby pushing the blood through the valve(s).

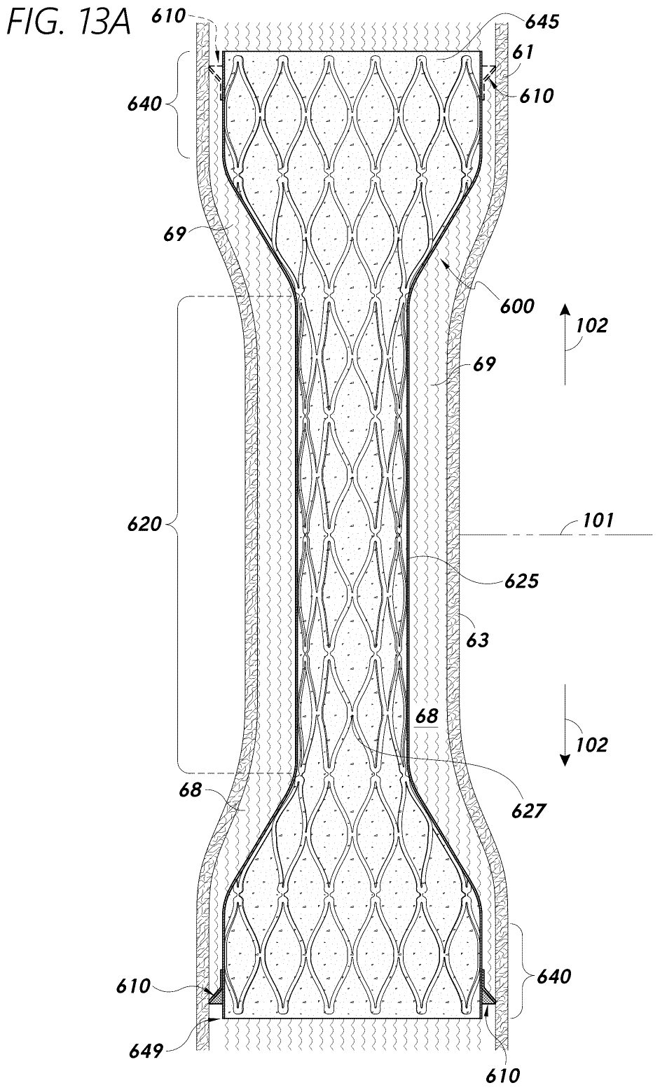

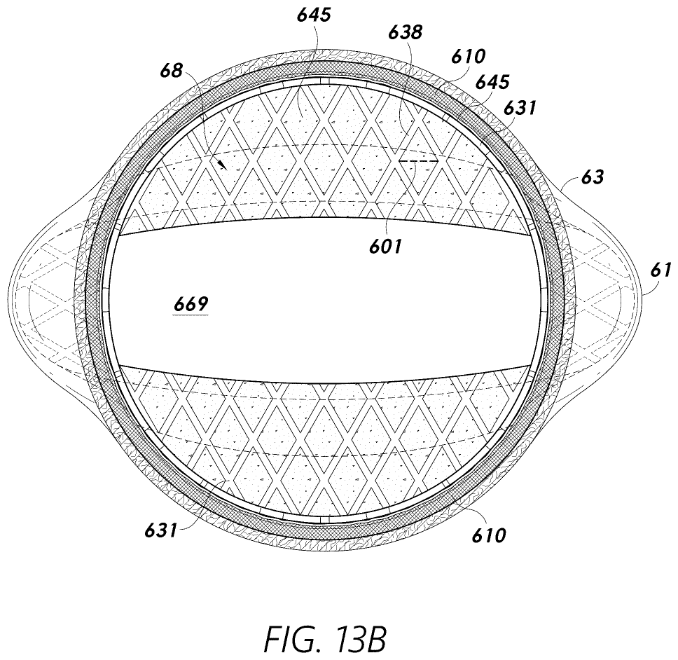

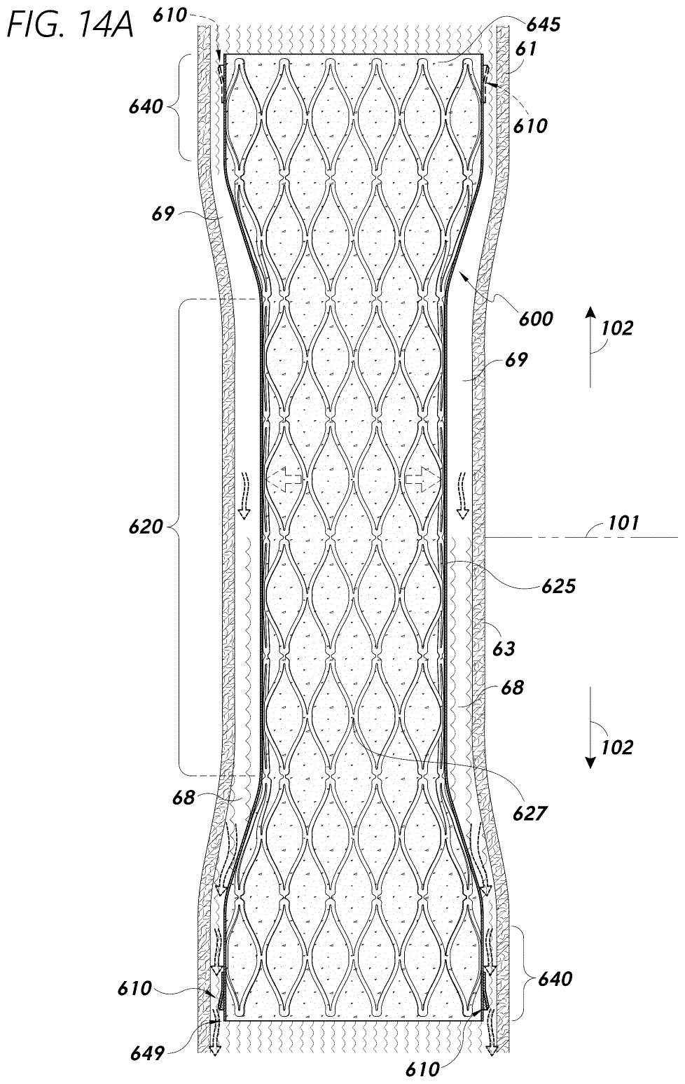

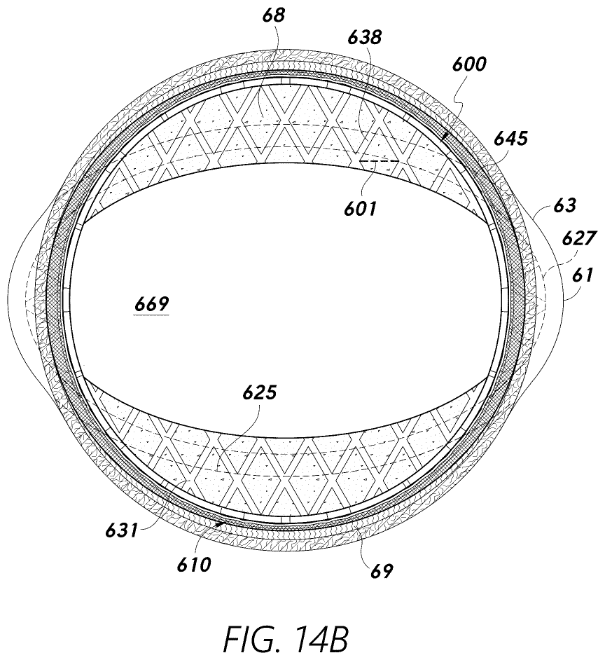

FIGS. 13A and 13B show side and axial views, respectively, of a stent implant device 600 including fluid expulsion valve(s) 610 in accordance with one or more examples. FIGS. 14A and 14B show side and axial views, respectively, of the stent implant device 600 having blood passed through expulsion valve(s) 610 associated therewith, which serve as egress channels for outflow of the trapped blood 69. The stent implant 600 may be a sealed/covered implant, as described in detail herein. Removal of the blood 69 through the egress channel(s)/valve(s) 110 can be via expulsion through expansion of minor-axis walls 625 of the stent 600.

The stent implant includes one or more one-way valves 610, which may be associated with one or both axial ends of the stent implant device 600. The one-way valve(s) 610 may be positioned and/or associated at/with the circular portions 640 of the stent implant 600, which may have a configuration as described in connection with any example disclosed herein. such as an implant having circular end portions configured to provide fluid sealing with the target blood vessel and a medial non-circular segment 620, which may have an oval shape or similar shape. The valve 610 may fully circumscribe the outer diameter of the end segment 640, or only a portion thereof.

The unidirectional valve(s) 610 may be disposed around at least one end 640 of the covered stent 600. The valve(s) 610 may be positioned between the stent's outer diameter/edge and the blood vessel's inner wall. The unidirectional valve(s) 610 can optionally surround the entire circumference of the stent segment(s) 640, or one or more areas/portions thereof. In some implementations, the valve(s) 640 span only, or primarily, minor-axis sides/portions of the stent 600.

The valve(s) 610 may be in fluid communication with blood 69 disposed in the gap area(s) 68 on an axially-inner side thereof. The blood 69 trapped between the stent's wall(s) and the vascular wall can exert fluid pressure on at least a portion of the valve(s) 610. Therefore, during systole or other pressurized condition, when the stent minor-axis wall(s) 625 expand outward, as shown in FIGS. 14A and 14B, the resulting elevated pressure in the trapped blood 69 may be sufficient to cause the valve(s) 610 to open and force at least a portion of the trapped blood 69 therethrough. Thus, each systolic cycle can serve to empty an additional volume of the trapped blood 69, which in-turn may draw the blood vessel wall 63 drawn closer to the stent 600, such as potentially up to full contact with the outer surface of the stent 600. One or more unidirectional valves 610 can be implemented around only one axial end of the stent, or one or more unidirectional valves can be implemented around both ends of the stent.

The one-way valve(s) 610 may have any suitable or desirable shape or configuration. For example, in some implementations, as shown in FIG. 13B, the valve 610 may extend along an outer diameter/perimeter of the circular portion 640 of the stent segment 640. Such valve(s) 640 may extend entirely around the perimeter of such segment(s), or around one or more arcs thereof. The valve(s) 610 may be concentric with the circular portion 640 of the stent segment 640. The valve(s) 610 may be configured as a one-way valve in that blood can pass from the gap area(s) 68 and out of the blood vessel segment spanned by the stent, but blood may be impeded/prevented from passing into the gap area(s) 68 from axially outside of the stent implant device 600. Therefore, once blood has been expelled from the gap area(s) 68, such gap areas may no longer be occupied by such volume of blood, thereby facilitating the collapse of the vacated gap area(s), which causes the blood vessel walls to form more closely to the stent walls and promotes reshaping functionality of the non-circular stent portion 620.

The one-way valve(s) 610 may comprise a flap configured to deflect in one axial direction and radially inwardly or outwardly, whereas deflection in the other axial direction beyond a certain point of contact with the blood vessel wall is prevented or impeded. For example, as pressure is increased in the gap area(s) 68, such pressure may cause blood 69 to push against the one-way valve(s) 610 and cause the blood 69 to pass therethrough, thereby expelling the blood 69 from the gap area(s) 68. Such increase in pressure may be achieved through any available means or mechanism. For example, as disclosed in detail herein, the medial portion 620 of the stent 600 may be expanded with respect to the minor-axis thereof, thereby reducing the gap area 68 and causing blood 69 occupying the gap area 68 to be pushed/expelled from around the stent 600 through the one-way valve(s) 610.

The one-way valve(s) 610 can have the form of one or more leaflets, skirts, flanges, lips, or the like. The valve(s) 610 can comprise any suitable or desirable at least partially flexible material, such as biocompatible rubber/polymer (e.g., polyurethane, silicone, etc.). The valve skirt 610 can be implemented at an angle that is axially deflected to produce the one-way opening range/functionality. The radial size of the valve(s) 610 can advantageously be limited to less than 20% of the diameter of the stent segment 640 to reduce interference with the channel area and/or sealing properties of the stent.

In some implementations, valve features 601 (see FIG. 13B) may be implemented in the cover 645, such as in slit(s)/cut(s) in the cover between struts 638 of the cells of the stent frame 631, to thereby allow for trapped blood to flow into the inner flow channel 669 of the stent 600 in response to increased pressure in the gap area(s) 68, such as in connection with systolic circularization of the stent segment 620. For example, leaflets or similar structures 601 implemented in the cover 645 or other portion/component of the stent 600 may be configured to open into the flow channel 669 of the stent 600 but at least partially impede or block back-flow through such features 601 into the gap area(s) 68.

### Gap Fluid Expulsion Using Balloon Inflation

The various solutions for removing trapped blood from around a non-circular stent segment, which can provide anti-thrombus functionality for stent implant devices, can include additional components associated with the stent implant device, such as tubes, valves, or the like, and/or can involve manipulation of the stent implant in a manner as to force trapped blood out from around the stent. Examples of such solutions can include/involve inflatable balloons, which can be inflated to deform/reshape the non-circular stent segment in a manner as to increase a minor-axis thereof, thereby circularizing the stent segment, which may decrease the gap area/volume between the stent segment and the blood vessel and force the blood occupying such space out from around the stent segment. In some examples, a balloon device may be utilized that has an ‘O,’ or donut, shape, which has a central aperture/opening through which blood can flow after inflation of the balloon, thereby preventing/reducing clogging of the blood vessel during balloon inflation/expansion.

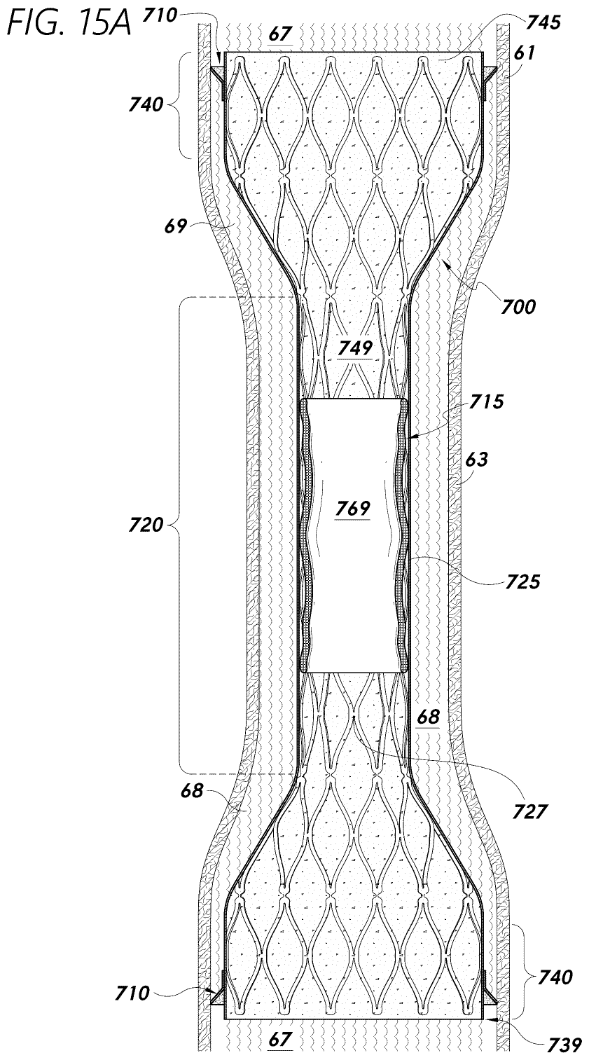

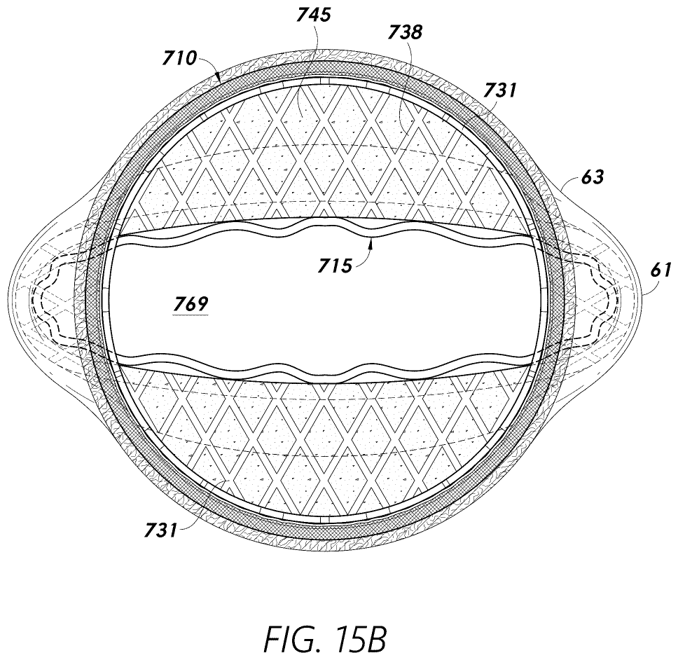

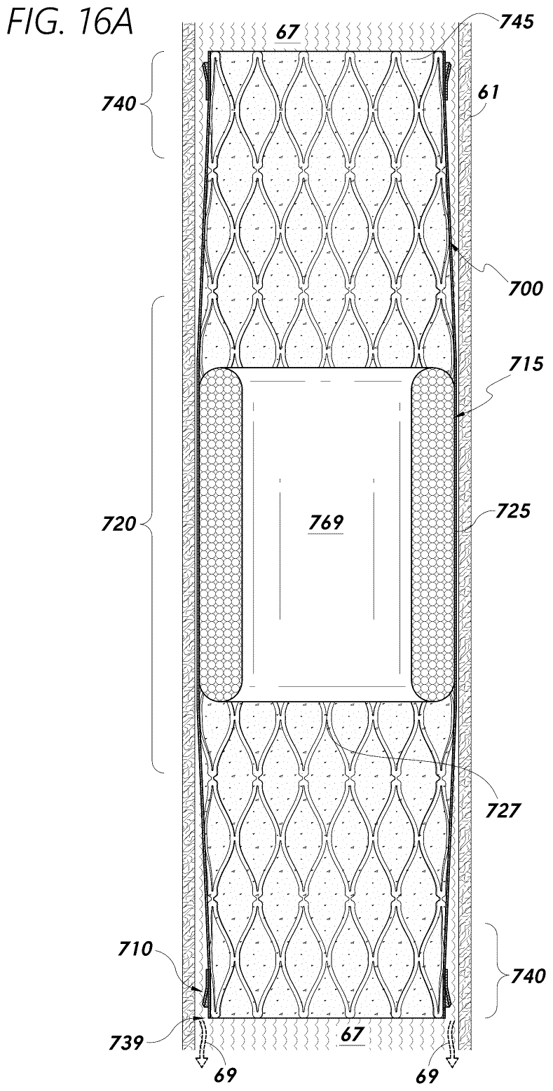

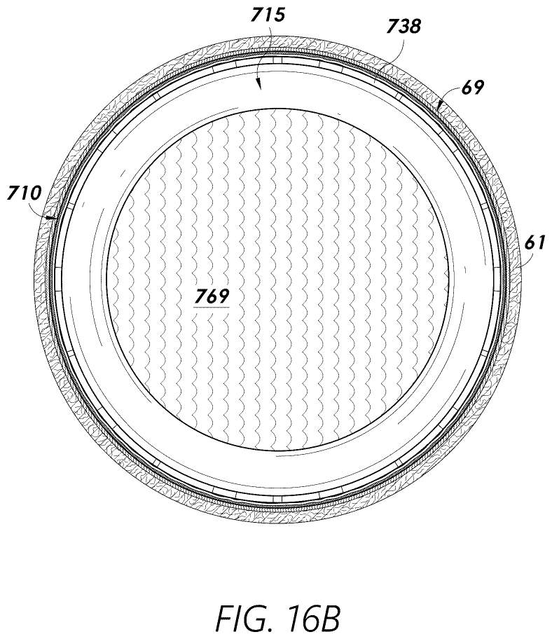

FIGS. 15A and 15B show side and axial views, respectively, of a stent implant device 700 having associated therewith a fluid expulsion balloon 715 in accordance with one or more examples. FIGS. 16A and 16B show side and axial views, respectively, of the stent implant device 700 with the fluid expulsion balloon 715 expanded in an internal channel 749 thereof in accordance with one or more examples. As described above, for stent implant devices that include fluid-tight coverings, blood expulsion/aspiration features as disclosed herein can reduce the risk of blood stagnation radially outside of the stent. For stent implant devices that do not include fluid-tight coverings, balloon and other expansion devices disclosed herein that are configured to circularize/expand non-circular stent segments can be utilized to couple the stent frame to the blood vessel wall in a manner as to allow for the blood vessel wall to serve as a blood flow channel that conforms to the shape/form of the stent frame.

The stent implant device 700 may be implemented in connection with a process that involves inflating the balloon 715 within the deployed covered stent 700, wherein expansion of the balloon 715 may force the trapped blood 69 through one or more unidirectional valves 710 or other flow-passage feature(s) associated with and/or disposed around one or both axial ends 740 of the stent 700. In the illustrated example, a unidirectional valve 710 as disclosed herein is provided around at least one end 740 (e.g., proximal or distal end) of the stent 700. For example, the valve(s) 710 may be disposed between the valve's outer diameter/edge and the blood vessel's inner wall. The unidirectional valve(s) 710 can optionally surround the entire circumference/perimeter of the stent segment(s) 740, or a portion thereof. The implant 700 can include valve features at both axial ends 740 or at only one axial end.

Alternative blood egress features providing passage from the gap area(s) 68 to the inner channel 749 of the stent 700 and/or to the area 67 axially beyond the stent 700 may be implemented within the scope of the present disclosure. Unlike some solutions disclosed herein, the unidirectional valve(s) may not be configured or designed to open in response to systolic blood pressure increase within the channel 749 and/or gap area(s) 68, but rather can be designed to open in response to a relatively higher pressure in the gap area(s) 68 imposed thereon by inflation of the balloon 715.

In some implementations, fluid communication between the opening/passage of the valve(s) 710 and the space 68 in which blood 69 is trapped between the stent wall and the vascular wall can be maintained, such that after stent deployment, the balloon 715 can be inflated within the stent channel 749, forcing the stent wall(s) to expand radially outward and push the blood 69 toward the valve(s) 710 at a pressure that will force the valve(s) 710 to open, allowing a volume of the trapped blood 69 (e.g., substantially all of the trapped blood 69; greater than 80% or 90% of the trapped blood 69) to be released into the blood stream within the channel 749 and/or in the area 67 axially outside of the stent 700.

FIGS. 15A and 15B show the balloon 715 in a deflated state prior to inflation thereof for trapped blood expulsion, whereas FIGS. 16A and 16B show a subsequent inflation of the balloon 715 and corresponding trapped blood expulsion. After inflation of the balloon 715 and expulsion of the blood 69, the balloon may again be deflated, thereby allowing the stent segment 720 to revert to its oval free/relaxed state, wherein the vascular wall may be drawn and/or more-closely conform to the non-circular shape of the stent segment 720 in the absence of the trapped blood volume 69 thereabout. For example, an at least partial vacuum may form between the stent and the blood vessel that draws/holds the blood vessel wall 63 to the stent wall(s) 725, thereby improving the coordination of the reshaping of the stent 720 with the reshaping of the blood vessel 61 to add compliance functionality thereto.

The balloon 715 can be provided, in some implementations, with an O-shaped cross-section (e.g., donut/torus, or cylindrical donut/torus, shape), defining a central opening/passageway 769 in its inflated state through which blood can continuously flow, thereby obviating the need for rapid pacing during the trapped blood expulsion procedure. Although an inflatable balloon is shown and described, in other examples, an internal temporary stent can be expanded within the oval stent segment 720 rather than a balloon. Such as temporary internal stent can be re-compressed and retrieved from the patient after expansion to expel the trapped blood 69, or the expansion stent can remain within the oval stent segment 720. For example, the expansion stent may be designed/configured to degrade over time and allow the oval stent segment 720 to revert to its oval free/relaxed state thereafter. The shape of the balloon 715 may be considered a cylindrical torus or donut, which forms an axial blood flow channel therethrough. The balloon 715, as shown, may be positioned in/within the inner diameter of the non-circular segment 320.

The implementation of the stent implant device 700 in FIGS. 15A, 15B, 16A, and 16B may include any suitable or desirable means or mechanism for expelling trapped blood from around the stent segment 720 through a blood flow passage, such as a one-way valve, tube, or other passage. That is, any of the examples disclosed herein may involve fluid expulsion using a balloon expandable device/component as shown in FIGS. 15A/15B and 16A/16B.

FIG. 15A shows the stent implant device 700 deployed in the target blood vessel 61 with the reshaping-expandable balloon 715 disposed within an inner diameter of the non-circular segment 720. The balloon 715 may have any suitable or desirable length and/or shape. For example, the balloon 715 may have a cylindrical shape, wherein in an expanded configuration thereof, the cylindrical balloon 715 has a generally circular axial cross-sectional shape. However, it should be understood that other shapes may be implemented within the scope of the present disclosure.

The stent 700 may have one or more donut-shaped balloons disposed within the frame of the stent. The balloon 715 can be physically attached/secured to the stent 700, or may be a separate component that is simply disposed/positioned within or the stent channel 749. The balloon(s) 715 can be inflatable, such that they may be inflated within the stent segment 720 to effect expansion and/or shaping of the stent segment 720 to conform to the shape of the inflated/expanded balloon(s) 715. Although one balloon 715 is shown, in some implementations, a plurality of axially-offset balloons may be implemented; stent re-shaping balloons may be positioned or associated with any axial position of the stent 700, and may further be arranged in any number of balloons and in any position or combination of positions. The balloon(s) 715 may be inflated such that a degree of inflation of the balloon(s) dictates, at least in part, the amount of blood expelled from the area(s) 68. The implementation of multiple axially-offset balloons can advantageously provide control over expansion in different shapes for different segments/areas of the stent 700 and/or in a sequential manner to promote movement of the blood 69 out of the trapped area(s) 68 and towards the axial ends 739

The balloon 715 can be a hollow, donut-shaped balloon, which can be inflated inside the stent 700 after deployment of the stent 700 in the target blood vessel 61. The donut-shape of the balloon 715 can permit blood-flow therethrough with relatively low flow disturbance, as blood may flow through a central flow channel 769 of the balloon 715.

The use of the expansion balloon(s) 715 can promote conformal contact between the stent 700 and the blood vessel wall, thereby facilitating tissue ingrowth and/or other attachment or secure frictional interference between the stent 700 and the blood vessel wall to secure the stent in-place in the target position in the blood vessel. The balloon(s) 715, individually or collectively, can span substantially the entire circumference and/or length of the stent 700, or only a portion of the circumference and/or length. The balloon(s) 715 may comprise any suitable or desirable material. For example, the balloon(s) 715 may comprise a compliant or non-compliant material (e.g., polymer). With respect to any blood-expulsion balloons disclosed herein, such balloons can advantageously comprise or be associated with one or more detachable inflation one-way nozzles that is/are fluidly coupled with/to an external delivery device that may be utilized for deployment purposes.

The balloon(s) 715 can be pre-attached to the stent 700 prior to deployment of the stent 700 in the blood vessel 68, or may be introduced into the lumen of the stent after deployment thereof. Furthermore, the balloon(s) 715 may be detachable from the stent 700 to allow for removal of the balloon(s) 715 after inflation for blood expulsion.

After removal of the blood 69, the balloon 715 may be deflated. Deflating of the balloon 715 may be implemented to allow for removal of the balloon 715 from the blood vessel 61. In some implementations, the balloon 715 may be maintained within the stent 700 after blood expulsion for an indefinite period after the implantation procedure. In such cases, deflation of the balloon 715 may reduce the volume thereof, thereby reducing the impact of the balloon 715 on blood flow within the vessel. The balloon 715 may be attached to the stent in some implementations, such as through suturing, adhesive, or other attachment means or mechanism.

The balloon 715 may be filled with any fluid medium, such as saline or other liquid, which may be preferable over gas medium due to the risk of stroke that may be associated with gas leakage in the event of balloon rupture or other leakage. The balloon 715 can be deployed and inflated as part of a separate procedure from the deployment of the stent 700. In some implementations, the balloon 715 is used to expand the stent 700 from a crimped/compressed delivery configuration thereof when deployed from the delivery system. Alternatively, the stent 700 may be self-expanding. It may be beneficial for the balloon 715 to be non-compliant in some instances as a means of controlling the expansion and/or shape of the balloon 715.

FIGS. 15A and 15B show the stent implant device 700 implanted in the target blood vessel segment, wherein the trapped blood 69 is present in the gap area(s) 68 outside of the outer diameter of the non-circular segment 720 of the stent 700. Due to the balloon 715 being in a deflated state, the balloon 715 may not initially reshape the stent 700 from its non-circular shape. In some implementations, the implant 700 may be deployed with the balloon 715 disposed therein, or alternatively the balloon may be introduced into the lumen of the stent segment 720 after deployment thereof.

FIG. 16A and 16B show the balloon 715 expanded to cause circularization of the stent segment 720, thereby reducing the volume of the gap area(s) 68 and forcing the blood 69 previously occupying such space 68 out from around the stent 700, thereby causing the blood vessel 61 to conform to the shape of the stent segment 720. When expanded, the full length of the stent 700 may form a straight cylindrical shape, as opposed to the combined circular and non-circular shape shown in FIGS. 15A. The blood 69 may exit the gap area(s) 68 through a one-way valve or tube passage 710, as described in detail herein, wherein blood may be impeded or prevented from reentry outside of the stent frame to the space on the outer diameter of the stent implant.

### ADDITIONAL EXAMPLES

Provided below is a list of examples, each of which may include aspects of any of the other examples disclosed herein. Furthermore, aspects of any example described above may be implemented in any of the numbered examples provided below.

Example 1: A stent comprising a first stent segment having a non-circular axial cross-sectional shape in a relaxed configuration thereof, second and third stent segments on opposite sides of the first stent segment, the second and third stent segments having a circular relaxed axial cross-sectional shape, a fluid-tight covering disposed on at least one of an outer diameter or an inner diameter of the first, second, and third stent segments, and a blood flow egress channel in fluid communication with a space on the outer diameter of the first stent segment, the blood flow egress channel configured to permit blood flow to pass axially out of the space through the blood flow egress channel.

Example 2: The stent of any example herein, in particular example 1, wherein the blood flow egress channel comprises a tube positioned on the outer diameter of the second stent segment.

Example 3: The stent of any example herein, in particular example 2, wherein the tube is coupled to an aspiration pump configured to aspirate blood through the tube.

Example 4: The stent of any example herein, in particular example 3, wherein the aspiration pump is associated with a delivery system configured to be used to deploy the stent in a target blood vessel.

Example 5: The stent of any example herein, in particular example 2, wherein the tube has a one-way valve in a flow channel thereof.

Example 6: The stent of any example herein, in particular example 2, wherein an axially-inner end of the tube deflects radially inwardly towards the outer diameter of the first stent segment.

Example 7: The stent of any example herein, in particular example 1, wherein the blood flow egress channel comprises a one-way valve configured to permit blood flow in a first axial direction through the one-way valve and block blood flow in a second axial direction opposite the first axial direction.

Example 8: The stent of any example herein, in particular example 7, wherein the one-way valve comprises a flap configured to deflect away from an axial center of the stent.

Example 9: The stent of any example herein, in particular example 7, wherein the one-way valve fully circumscribes the outer diameter of the second stent segment.

Example 10: The stent of any example herein, in particular example 1, wherein the blood flow egress channel comprises an opening in the fluid-tight covering.

Example 11: The stent of any example herein, in particular example 10, wherein the opening in the fluid-tight covering is in a portion of the fluid-tight covering associated with a transition segment of the stent between the first stent segment and the second stent segment.

Example 12: The stent of any example herein, in particular example 1, further comprising an inflatable balloon disposed within the inner diameter of the first stent segment.

Example 13: The stent of any example herein, in particular example 12, wherein the balloon has an axial blood flow channel.

Example 14: A method of removing trapped blood from around a stent implant, the method comprising deploying a covered stent implant in a blood vessel, the stent implant including a medial non-circular stent segment, causing a first and second circular end segments of the stent implant to expand to fluidly seal the first and second circular end segments against the blood vessel, and removing trapped blood from a space between an outer diameter of the medial non-circular stent segment and the blood vessel.

Example 15: The method of any example herein, in particular example 14, wherein said removing the trapped blood comprises aspirating the trapped blood through a tube that runs along an outer diameter of the first circular end segment.

Example 16: The method of any example herein, in particular example 14, wherein said removing the trapped blood comprises expelling the trapped blood from the space by causing expansion of one or more minor-axis walls of the medial non-circular stent segment.

Example 17: The method of any example herein, in particular example 16, wherein said expelling the trapped blood comprises pushing the trapped blood through a tube that runs along an outer diameter of the first circular end segment.

Example 18: The method of any example herein, in particular example 17, wherein the tube comprises a one-way valve in a flow channel of the tube.

Example 19: The method of any example herein, in particular example 16, wherein said expelling the trapped blood comprises pushing the trapped blood through a one-way valve associated with the first circular end segment.

Example 20: The method of any example herein, in particular example 19, wherein the one-way valve comprises a flexible flange disposed between an outer diameter of the first circular end segment and the blood vessel.

Example 21: The method of any example herein, in particular example 16, wherein said expelling the trapped blood comprises inflating a balloon within a channel of the medial non-circular stent segment.

Example 22: The method of any example herein, in particular example 21, wherein the balloon has a cylindrical torus form that forms an axial blood flow channel therethrough.

Example 23: The method of any example herein, in particular example 22, further comprising, after said inflating the balloon, deflating and removing the balloon from the blood vessel.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising.” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term. element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example. Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for case of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A method of evacuating blood from around a stent implant to promote adhesive attachment between the stent and the blood vessel, the method comprising:
deploying a covered stent implant in a blood vessel, the stent implant including a medial non-circular stent segment;
causing a first and second circular end segments of the stent implant to expand to fluidly seal the first and second circular end segments against the blood vessel; and
removing trapped blood from a space between an outer diameter of the medial non-circular stent segment and the blood vessel.

2. The method of claim 1, wherein said removing the trapped blood comprises aspirating the trapped blood through a tube that runs along an outer diameter of the first circular end segment.

3. The method of claim 1, wherein said removing the trapped blood comprises expelling the trapped blood from the space by causing expansion of one or more minor-axis walls of the medial non-circular stent segment.

4. The method of claim 3, wherein said expelling the trapped blood comprises pushing the trapped blood through a tube that runs along an outer diameter of the first circular end segment.

5. The method of claim 4, wherein the tube comprises a one-way valve in a flow channel of the tube.

6. The method of claim 3, wherein said expelling the trapped blood comprises pushing the trapped blood through a one-way valve associated with the first circular end segment.

7. The method of claim 6, wherein the one-way valve comprises a flexible flange disposed between an outer diameter of the first circular end segment and the blood vessel.

8. The method of claim 3, wherein said expelling the trapped blood comprises inflating a balloon within a channel of the medial non-circular stent segment.

9. The method of claim 8, wherein the balloon has a cylindrical torus form that forms an axial blood flow channel therethrough.

10. The method of claim 9, further comprising, after said inflating the balloon, deflating and removing the balloon from the blood vessel.

11. A method of removing trapped blood from around a stent implant, the method comprising:
providing a stent implant device including:
a first stent segment having a non-circular axial cross-sectional shape in a relaxed configuration thereof;
second and third stent segments on opposite sides of the first stent segment, the second and third stent segments having a circular relaxed axial cross-sectional shape;
a fluid-tight covering disposed on at least one of an outer diameter or an inner diameter of the first, second, and third stent segments; and
a blood flow egress channel in fluid communication with a space on the outer diameter of the first stent segment;
deploying the stent implant in a target blood vessel segment, thereby trapping a portion of blood in the space on the outer diameter of the first stent segment between the second and third stent segments; and
passing the trapped portion of blood axially out of the space through the blood flow egress channel.

12. The method of claim 11, wherein passing the trapped portion of blood involves aspirating the trapped portion of blood through the blood flow egress channel using an aspiration pump.

13. The method of claim 11, wherein passing the trapped portion of blood involves inflating a balloon within the inner diameter of the first stent segment, the balloon having an axial blood flow channel.

14. The method of claim 11, wherein the blood flow egress channel comprises a tube positioned on the outer diameter of the second stent segment.

15. The method of claim 14, wherein the tube has a one-way valve in a flow channel thereof.

16. The method of claim 14, wherein an axially-inner end of the tube deflects radially inwardly towards the outer diameter of the first stent segment.

17. The method of claim 11, wherein the blood flow egress channel comprises a one-way valve adapted to permit blood flow in a first axial direction through the one-way valve and block blood flow in a second axial direction opposite the first axial direction.

18. The method of claim 11, wherein the blood flow egress channel comprises an opening in the fluid-tight covering.

19. The method of claim 18, wherein the opening in the fluid-tight covering is in a portion of the fluid-tight covering associated with a transition segment of the stent between the first stent segment and the second stent segment.

20. A method of removing trapped blood from around a stent implant, the method comprising:
deploying a covered stent implant in a blood vessel, the stent implant including a medial oval-shaped segment;
expanding first and second circle-shaped end segments of the stent implant within the blood vessel to fluidly seal the first and second circle-shaped end segments against the blood vessel and trap a portion of blood in a space radially between minor-axis sidewall portions of the medial oval-shaped segment and an inner diameter of the blood vessel; and
removing the trapped portion of blood from the space by passing the trapped portion of blood between an outside of the first circle-shaped end segment and the inner diameter of the blood vessel.
